SEQUENCE LISTING (325 sequences)

SEQ ID NO: 1 - DNA, 509 bp, Homo sapiens
  CDS (13)..(498)
aggagggccaccatgcgaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgattgcaatt

SEQ ID NO: 2 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 3 - DNA, 35 bp, artificial sequence
  primer
tcaatctatgcatattgcggctactttatatacgg

SEQ ID NO: 4 - DNA, 35 bp, artificial sequence
  primer
tcaatctatgcatattttcgctactttatatacgg

SEQ ID NO: 5 - DNA, 35 bp, artificial sequence
  primer
tcaatctatgcatattattgctactttatatacgg

SEQ ID NO: 6 - DNA, 35 bp, artificial sequence
  primer
tcaatctatgcatattctggctactttatatacgg

SEQ ID NO: 7 - DNA, 35 bp, artificial sequence
  primer
tcaatctatgcatattccggctactttatatacgg

SEQ ID NO: 8 - DNA, 35 bp, artificial sequence
  primer
tcaatctatgcatattgtggctactttatatacgg

SEQ ID NO: 9 - DNA, 35 bp, artificial sequence
  primer
tcaatctatgcatatttgggctactttatatacgg

SEQ ID NO: 10 - DNA, 35 bp, artificial sequence
  primer
tcaatctatgcatattatggctactttatatacgg

SEQ ID NO: 11 - DNA, 35 bp, artificial sequence
  primer
ttcgaaaccacatttggcgagtatttccatccagt

SEQ ID NO: 12 - DNA, 35 bp, artificial sequence
  primer
ttcgaaaccacatttgttcagtatttccatccagt

SEQ ID NO: 13 - DNA, 35 bp, artificial sequence
  primer
ttcgaaaccacatttgattagtatttccatccagt

SEQ ID NO: 14 - DNA, 35 bp, artificial sequence
  primer
ttcgaaaccacatttgctgagtatttccatccagt

SEQ ID NO: 15 - DNA, 35 bp, artificial sequence
  primer
ttcgaaaccacatttgccgagtatttccatccagt

SEQ ID NO: 16 - DNA, 35 bp, artificial sequence
  primer
ttcgaaaccacatttggtgagtatttccatccagt

SEQ ID NO: 17 - DNA, 35 bp, artificial sequence
  primer
ttcgaaaccacatttgtggagtatttccatccagt

SEQ ID NO: 18 - DNA, 35 bp, artificial sequence
  primer
ttcgaaaccacatttgatgagtatttccatccagt

SEQ ID NO: 19 - DNA, 35 bp, artificial sequence
  primer
tcattttctaactgaagcgggcattcatgtcttca

SEQ ID NO: 20 - DNA, 35 bp, artificial sequence
  primer
tcattttctaactgaattcggcattcatgtcttca

SEQ ID NO: 21 - DNA, 35 bp, artificial sequence
  primer
tcattttctaactgaaattggcattcatgtcttca

SEQ ID NO: 22 - DNA, 35 bp, artificial sequence
  primer
tcattttctaactgaactgggcattcatgtcttca

SEQ ID NO: 23 - DNA, 35 bp, artificial sequence
  primer
tcattttctaactgaaccgggcattcatgtcttca

SEQ ID NO: 24 - DNA, 35 bp, artificial sequence
  primer
tcattttctaactgaagtgggcattcatgtcttca

SEQ ID NO: 25 - DNA, 35 bp, artificial sequence
  primer
tcattttctaactgaatggggcattcatgtcttca

SEQ ID NO: 26 - DNA, 35 bp, artificial sequence
  primer
tcattttctaactgaaatgggcattcatgtcttca

SEQ ID NO: 27 - DNA, 35 bp, artificial sequence
  primer
ttttctaactgaagctgcgattcatgtcttcattt

SEQ ID NO: 28 - DNA, 35 bp, artificial sequence
  primer
ttttctaactgaagctttcattcatgtcttcattt

SEQ ID NO: 29 - DNA, 35 bp, artificial sequence
  primer
ttttctaactgaagctattattcatgtcttcattt

SEQ ID NO: 30 - DNA, 35 bp, artificial sequence
  primer
ttttctaactgaagctctgattcatgtcttcattt

SEQ ID NO: 31 - DNA, 35 bp, artificial sequence
  primer
ttttctaactgaagctccgattcatgtcttcattt

SEQ ID NO: 32 - DNA, 35 bp, artificial sequence
  primer
ttttctaactgaagctgtgattcatgtcttcattt

SEQ ID NO: 33 - DNA, 35 bp, artificial sequence
  primer
ttttctaactgaagcttggattcatgtcttcattt

SEQ ID NO: 34 - DNA, 35 bp, artificial sequence
  primer
ttttctaactgaagctatgattcatgtcttcattt

SEQ ID NO: 35 - DNA, 35 bp, artificial sequence
  primer
tttgaaaaaaattgaagcgcttattcaatctatgc

SEQ ID NO: 36 - DNA, 35 bp, artificial sequence
  primer
tttgaaaaaaattgaattccttattcaatctatgc

SEQ ID NO: 37 - DNA, 35 bp, artificial sequence
  primer
tttgaaaaaaattgaaattcttattcaatctatgc

SEQ ID NO: 38 - DNA, 35 bp, artificial sequence
  primer
tttgaaaaaaattgaactgcttattcaatctatgc

SEQ ID NO: 39 - DNA, 35 bp, artificial sequence
  primer
tttgaaaaaaattgaaccgcttattcaatctatgc

SEQ ID NO: 40 - DNA, 35 bp, artificial sequence
  primer
tttgaaaaaaattgaagtgcttattcaatctatgc

SEQ ID NO: 41 - DNA, 35 bp, artificial sequence
  primer
tttgaaaaaaattgaatggcttattcaatctatgc

SEQ ID NO: 42 - DNA, 35 bp, artificial sequence
  primer
tttgaaaaaaattgaaatgcttattcaatctatgc

SEQ ID NO: 43 - DNA, 35 bp, artificial sequence
  primer
gttacaagttatttcagcggagtccggagatgcaa

SEQ ID NO: 44 - DNA, 35 bp, artificial sequence
  primer
gttacaagttatttcattcgagtccggagatgcaa

SEQ ID NO: 45 - DNA, 35 bp, artificial sequence
  primer
gttacaagttatttcaattgagtccggagatgcaa

SEQ ID NO: 46 - DNA, 35 bp, artificial sequence
  primer
gttacaagttatttcactggagtccggagatgcaa

SEQ ID NO: 47 - DNA, 35 bp, artificial sequence
  primer
gttacaagttatttcaccggagtccggagatgcaa

SEQ ID NO: 48 - DNA, 35 bp, artificial sequence
  primer
gttacaagttatttcagtggagtccggagatgcaa

SEQ ID NO: 49 - DNA, 35 bp, artificial sequence
  primer
gttacaagttatttcatgggagtccggagatgcaa

SEQ ID NO: 50 - DNA, 35 bp, artificial sequence
  primer
gttacaagttatttcaatggagtccggagatgcaa

SEQ ID NO: 51 - DNA, 35 bp, artificial sequence
  primer
tcttattcaatctatggcgattgatgctactttat

SEQ ID NO: 52 - DNA, 35 bp, artificial sequence
  primer
tcttattcaatctatgttcattgatgctactttat

SEQ ID NO: 53 - DNA, 35 bp, artificial sequence
  primer
tcttattcaatctatgattattgatgctactttat

SEQ ID NO: 54 - DNA, 35 bp, artificial sequence
  primer
tcttattcaatctatgctgattgatgctactttat

SEQ ID NO: 55 - DNA, 35 bp, artificial sequence
  primer
tcttattcaatctatgccgattgatgctactttat

SEQ ID NO: 56 - DNA, 35 bp, artificial sequence
  primer
tcttattcaatctatggtgattgatgctactttat

SEQ ID NO: 57 - DNA, 35 bp, artificial sequence
  primer
tcttattcaatctatgtggattgatgctactttat

SEQ ID NO: 58 - DNA, 35 bp, artificial sequence
  primer
tcttattcaatctatgatgattgatgctactttat

SEQ ID NO: 59 - DNA, 35 bp, artificial sequence
  primer
cagttgcaaagtaacagcgatgaagtgctttctct

SEQ ID NO: 60 - DNA, 35 bp, artificial sequence
  primer
cagttgcaaagtaacattcatgaagtgctttctct

SEQ ID NO: 61 - DNA, 35 bp, artificial sequence
  primer
cagttgcaaagtaacaattatgaagtgctttctct

SEQ ID NO: 62 - DNA, 35 bp, artificial sequence
  primer
cagttgcaaagtaacactgatgaagtgctttctct

SEQ ID NO: 63 - DNA, 35 bp, artificial sequence
  primer
cagttgcaaagtaacaccgatgaagtgctttctct

SEQ ID NO: 64 - DNA, 35 bp, artificial sequence
  primer
cagttgcaaagtaacagtgatgaagtgctttctct

SEQ ID NO: 65 - DNA, 35 bp, artificial sequence
  primer
cagttgcaaagtaacatggatgaagtgctttctct

SEQ ID NO: 66 - DNA, 35 bp, artificial sequence
  primer
cagttgcaaagtaacaatgatgaagtgctttctct

SEQ ID NO: 67 - DNA, 35 bp, artificial sequence
  primer
cggagatgcaagtattgcggatacagtagaaaatc

SEQ ID NO: 68 - DNA, 35 bp, artificial sequence
  primer
cggagatgcaagtattttcgatacagtagaaaatc

SEQ ID NO: 69 - DNA, 35 bp, artificial sequence
  primer
cggagatgcaagtattattgatacagtagaaaatc

SEQ ID NO: 70 - DNA, 35 bp, artificial sequence
  primer
cggagatgcaagtattctggatacagtagaaaatc

SEQ ID NO: 71 - DNA, 35 bp, artificial sequence
  primer
cggagatgcaagtattccggatacagtagaaaatc

SEQ ID NO: 72 - DNA, 35 bp, artificial sequence
  primer
cggagatgcaagtattgtggatacagtagaaaatc

SEQ ID NO: 73 - DNA, 35 bp, artificial sequence
  primer
cggagatgcaagtatttgggatacagtagaaaatc

SEQ ID NO: 74 - DNA, 35 bp, artificial sequence
  primer
cggagatgcaagtattatggatacagtagaaaatc

SEQ ID NO: 75 - DNA, 35 bp, artificial sequence
  primer
agaaaatctgatcatcgcggcaaacaacagtttgt

SEQ ID NO: 76 - DNA, 35 bp, artificial sequence
  primer
agaaaatctgatcatcttcgcaaacaacagtttgt

SEQ ID NO: 77 - DNA, 35 bp, artificial sequence
  primer
agaaaatctgatcatcattgcaaacaacagtttgt

SEQ ID NO: 78 - DNA, 35 bp, artificial sequence
  primer
agaaaatctgatcatcctggcaaacaacagtttgt

SEQ ID NO: 79 - DNA, 35 bp, artificial sequence
  primer
agaaaatctgatcatcccggcaaacaacagtttgt

SEQ ID NO: 80 - DNA, 35 bp, artificial sequence
  primer
agaaaatctgatcatcgtggcaaacaacagtttgt

SEQ ID NO: 81 - DNA, 35 bp, artificial sequence
  primer
agaaaatctgatcatctgggcaaacaacagtttgt

SEQ ID NO: 82 - DNA, 35 bp, artificial sequence
  primer
agaaaatctgatcatcatggcaaacaacagtttgt

SEQ ID NO: 83 - DNA, 35 bp, artificial sequence
  primer
ggaactggaggaaaaagcgattaaagaatttttgc

SEQ ID NO: 84 - DNA, 35 bp, artificial sequence
  primer
ggaactggaggaaaaattcattaaagaatttttgc

SEQ ID NO: 85 - DNA, 35 bp, artificial sequence
  primer
ggaactggaggaaaaaattattaaagaatttttgc

SEQ ID NO: 86 - DNA, 35 bp, artificial sequence
  primer
ggaactggaggaaaaactgattaaagaatttttgc

SEQ ID NO: 87 - DNA, 35 bp, artificial sequence
  primer
ggaactggaggaaaaaccgattaaagaatttttgc

SEQ ID NO: 88 - DNA, 35 bp, artificial sequence
  primer
ggaactggaggaaaaagtgattaaagaatttttgc

SEQ ID NO: 89 - DNA, 35 bp, artificial sequence
  primer
ggaactggaggaaaaatggattaaagaatttttgc

SEQ ID NO: 90 - DNA, 35 bp, artificial sequence
  primer
ggaactggaggaaaaaatgattaaagaatttttgc

SEQ ID NO: 91 - DNA, 35 bp, artificial sequence
  primer
atttttgcagagttttgcgcatattgtccaaatgt

SEQ ID NO: 92 - DNA, 35 bp, artificial sequence
  primer
atttttgcagagttttttccatattgtccaaatgt

SEQ ID NO: 93 - DNA, 35 bp, artificial sequence
  primer
atttttgcagagttttattcatattgtccaaatgt

SEQ ID NO: 94 - DNA, 35 bp, artificial sequence
  primer
atttttgcagagttttctgcatattgtccaaatgt

SEQ ID NO: 95 - DNA, 35 bp, artificial sequence
  primer
atttttgcagagttttccgcatattgtccaaatgt

SEQ ID NO: 96 - DNA, 35 bp, artificial sequence
  primer
atttttgcagagttttgtgcatattgtccaaatgt

SEQ ID NO: 97 - DNA, 35 bp, artificial sequence
  primer
atttttgcagagtttttggcatattgtccaaatgt

SEQ ID NO: 98 - DNA, 35 bp, artificial sequence
  primer
atttttgcagagttttatgcatattgtccaaatgt

SEQ ID NO: 99 - DNA, 35 bp, artificial sequence
  primer
tttgcagagttttgtagcgattgtccaaatgttca

SEQ ID NO: 100 - DNA, 35 bp, artificial sequence
  primer
tttgcagagttttgtattcattgtccaaatgttca

SEQ ID NO: 101 - DNA, 35 bp, artificial sequence
  primer
tttgcagagttttgtaattattgtccaaatgttca

SEQ ID NO: 102 - DNA, 35 bp, artificial sequence
  primer
tttgcagagttttgtactgattgtccaaatgttca

SEQ ID NO: 103 - DNA, 35 bp, artificial sequence
  primer
tttgcagagttttgtaccgattgtccaaatgttca

SEQ ID NO: 104 - DNA, 35 bp, artificial sequence
  primer
tttgcagagttttgtagtgattgtccaaatgttca

SEQ ID NO: 105 - DNA, 35 bp, artificial sequence
  primer
tttgcagagttttgtatggattgtccaaatgttca

SEQ ID NO: 106 - DNA, 35 bp, artificial sequence
  primer
tttgcagagttttgtaatgattgtccaaatgttca

SEQ ID NO: 107 - DNA, 35 bp, artificial sequence
  primer
agttatttcacttgaggcgggagatgcaagtattc

SEQ ID NO: 108 - DNA, 35 bp, artificial sequence
  primer
agttatttcacttgagttcggagatgcaagtattc

SEQ ID NO: 109 - DNA, 35 bp, artificial sequence
  primer
agttatttcacttgagattggagatgcaagtattc

SEQ ID NO: 110 - DNA, 35 bp, artificial sequence
  primer
agttatttcacttgagctgggagatgcaagtattc

SEQ ID NO: 111 - DNA, 35 bp, artificial sequence
  primer
agttatttcacttgagccgggagatgcaagtattc

SEQ ID NO: 112 - DNA, 35 bp, artificial sequence
  primer
agttatttcacttgaggtgggagatgcaagtattc

SEQ ID NO: 113 - DNA, 35 bp, artificial sequence
  primer
agttatttcacttgagtggggagatgcaagtattc

SEQ ID NO: 114 - DNA, 35 bp, artificial sequence
  primer
agttatttcacttgagatgggagatgcaagtattc

SEQ ID NO: 115 - DNA, 35 bp, artificial sequence
  primer
taatgggaatgtaacagcgtctggatgcaaagaat

SEQ ID NO: 116 - DNA, 35 bp, artificial sequence
  primer
taatgggaatgtaacattctctggatgcaaagaat

SEQ ID NO: 117 - DNA, 35 bp, artificial sequence
  primer
taatgggaatgtaacaatttctggatgcaaagaat

SEQ ID NO: 118 - DNA, 35 bp, artificial sequence
  primer
taatgggaatgtaacactgtctggatgcaaagaat

SEQ ID NO: 119 - DNA, 35 bp, artificial sequence
  primer
taatgggaatgtaacaccgtctggatgcaaagaat

SEQ ID NO: 120 - DNA, 35 bp, artificial sequence
  primer
taatgggaatgtaacagtgtctggatgcaaagaat

SEQ ID NO: 121 - DNA, 35 bp, artificial sequence
  primer
taatgggaatgtaacatggtctggatgcaaagaat

SEQ ID NO: 122 - DNA, 35 bp, artificial sequence
  primer
taatgggaatgtaacaatgtctggatgcaaagaat

SEQ ID NO: 123 - DNA, 35 bp, artificial sequence
  primer
ggaggaaaaaaatattgcggaatttttgcagagtt

SEQ ID NO: 124 - DNA, 35 bp, artificial sequence
  primer
ggaggaaaaaaatattttcgaatttttgcagagtt

SEQ ID NO: 125 - DNA, 35 bp, artificial sequence
  primer
ggaggaaaaaaatattattgaatttttgcagagtt

SEQ ID NO: 126 - DNA, 35 bp, artificial sequence
  primer
ggaggaaaaaaatattctggaatttttgcagagtt

SEQ ID NO: 127 - DNA, 35 bp, artificial sequence
  primer
ggaggaaaaaaatattccggaatttttgcagagtt

SEQ ID NO: 128 - DNA, 35 bp, artificial sequence
  primer
ggaggaaaaaaatattgtggaatttttgcagagtt

SEQ ID NO: 129 - DNA, 35 bp, artificial sequence
  primer
ggaggaaaaaaatatttgggaatttttgcagagtt

SEQ ID NO: 130 - DNA, 35 bp, artificial sequence
  primer
ggaggaaaaaaatattatggaatttttgcagagtt

SEQ ID NO: 131 - DNA, 35 bp, artificial sequence
  primer
acaagttatttcacttgcgtccggagatgcaagta

SEQ ID NO: 132 - DNA, 35 bp, artificial sequence
  primer
acaagttatttcacttttctccggagatgcaagta

SEQ ID NO: 133 - DNA, 35 bp, artificial sequence
  primer
acaagttatttcacttatttccggagatgcaagta

SEQ ID NO: 134 - DNA, 35 bp, artificial sequence
  primer
acaagttatttcacttctgtccggagatgcaagta

SEQ ID NO: 135 - DNA, 35 bp, artificial sequence
  primer
acaagttatttcacttccgtccggagatgcaagta

SEQ ID NO: 136 - DNA, 35 bp, artificial sequence
  primer
acaagttatttcacttgtgtccggagatgcaagta

SEQ ID NO: 137 - DNA, 35 bp, artificial sequence
  primer
acaagttatttcactttggtccggagatgcaagta

SEQ ID NO: 138 - DNA, 35 bp, artificial sequence
  primer
acaagttatttcacttatgtccggagatgcaagta

SEQ ID NO: 139 - DNA, 35 bp, artificial sequence
  primer
ggccaccatgcgaattgcgaaaccacatttgagaa

SEQ ID NO: 140 - DNA, 35 bp, artificial sequence
  primer
ggccaccatgcgaattttcaaaccacatttgagaa

SEQ ID NO: 141 - DNA, 35 bp, artificial sequence
  primer
ggccaccatgcgaattattaaaccacatttgagaa

SEQ ID NO: 142 - DNA, 35 bp, artificial sequence
  primer
ggccaccatgcgaattctgaaaccacatttgagaa

SEQ ID NO: 143 - DNA, 35 bp, artificial sequence
  primer
ggccaccatgcgaattccgaaaccacatttgagaa

SEQ ID NO: 144 - DNA, 35 bp, artificial sequence
  primer
ggccaccatgcgaattgtgaaaccacatttgagaa

SEQ ID NO: 145 - DNA, 35 bp, artificial sequence
  primer
ggccaccatgcgaatttggaaaccacatttgagaa

SEQ ID NO: 146 - DNA, 35 bp, artificial sequence
  primer
ggccaccatgcgaattatgaaaccacatttgagaa

SEQ ID NO: 147 - DNA, 35 bp, artificial sequence
  primer
tcctaaaacagaagccgcgtgggtgaatgtaataa

SEQ ID NO: 148 - DNA, 35 bp, artificial sequence
  primer
tcctaaaacagaagccttctgggtgaatgtaataa

SEQ ID NO: 149 - DNA, 35 bp, artificial sequence
  primer
tcctaaaacagaagccatttgggtgaatgtaataa

SEQ ID NO: 150 - DNA, 35 bp, artificial sequence
  primer
tcctaaaacagaagccctgtgggtgaatgtaataa

SEQ ID NO: 151 - DNA, 35 bp, artificial sequence
  primer
tcctaaaacagaagccccgtgggtgaatgtaataa

SEQ ID NO: 152 - DNA, 35 bp, artificial sequence
  primer
tcctaaaacagaagccgtgtgggtgaatgtaataa

SEQ ID NO: 153 - DNA, 35 bp, artificial sequence
  primer
tcctaaaacagaagcctggtgggtgaatgtaataa

SEQ ID NO: 154 - DNA, 35 bp, artificial sequence
  primer
tcctaaaacagaagccatgtgggtgaatgtaataa

SEQ ID NO: 155 - DNA, 35 bp, artificial sequence
  primer
tgtccaaatgttcatcgcgacttcttgattgcaat

SEQ ID NO: 156 - DNA, 35 bp, artificial sequence
  primer
tgtccaaatgttcatcttcacttcttgattgcaat

SEQ ID NO: 157 - DNA, 35 bp, artificial sequence
  primer
tgtccaaatgttcatcattacttcttgattgcaat

SEQ ID NO: 158 - DNA, 35 bp, artificial sequence
  primer
tgtccaaatgttcatcctgacttcttgattgcaat

SEQ ID NO: 159 - DNA, 35 bp, artificial sequence
  primer
tgtccaaatgttcatcccgacttcttgattgcaat

SEQ ID NO: 160 - DNA, 35 bp, artificial sequence
  primer
tgtccaaatgttcatcgtgacttcttgattgcaat

SEQ ID NO: 161 - DNA, 35 bp, artificial sequence
  primer
tgtccaaatgttcatctggacttcttgattgcaat

SEQ ID NO: 162 - DNA, 35 bp, artificial sequence
  primer
tgtccaaatgttcatcatgacttcttgattgcaat

SEQ ID NO: 163 - DNA, 35 bp, artificial sequence
  primer
aattgaagatcttattgcgtctatgcatattgatg

SEQ ID NO: 164 - DNA, 35 bp, artificial sequence
  primer
aattgaagatcttattttctctatgcatattgatg

SEQ ID NO: 165 - DNA, 35 bp, artificial sequence
  primer
aattgaagatcttattatttctatgcatattgatg

SEQ ID NO: 166 - DNA, 35 bp, artificial sequence
  primer
aattgaagatcttattctgtctatgcatattgatg

SEQ ID NO: 167 - DNA, 35 bp, artificial sequence
  primer
aattgaagatcttattccgtctatgcatattgatg

SEQ ID NO: 168 - DNA, 35 bp, artificial sequence
  primer
aattgaagatcttattgtgtctatgcatattgatg

SEQ ID NO: 169 - DNA, 35 bp, artificial sequence
  primer
aattgaagatcttatttggtctatgcatattgatg

SEQ ID NO: 170 - DNA, 35 bp, artificial sequence
  primer
aattgaagatcttattatgtctatgcatattgatg

SEQ ID NO: 171 - DNA, 35 bp, artificial sequence
  primer
tgctactttatatacggcgagtgatgttcacccca

SEQ ID NO: 172 - DNA, 35 bp, artificial sequence
  primer
tgctactttatatacgttcagtgatgttcacccca

SEQ ID NO: 173 - DNA, 35 bp, artificial sequence
  primer
tgctactttatatacgattagtgatgttcacccca

SEQ ID NO: 174 - DNA, 35 bp, artificial sequence
  primer
tgctactttatatacgctgagtgatgttcacccca

SEQ ID NO: 175 - DNA, 35 bp, artificial sequence
  primer
tgctactttatatacgccgagtgatgttcacccca

SEQ ID NO: 176 - DNA, 35 bp, artificial sequence
  primer
tgctactttatatacggtgagtgatgttcacccca

SEQ ID NO: 177 - DNA, 35 bp, artificial sequence
  primer
tgctactttatatacgtggagtgatgttcacccca

SEQ ID NO: 178 - DNA, 35 bp, artificial sequence
  primer
tgctactttatatacgatgagtgatgttcacccca

SEQ ID NO: 179 - DNA, 35 bp, artificial sequence
  primer
tcaccccagttgcaaagcgacagcaatgaagtgct

SEQ ID NO: 180 - DNA, 35 bp, artificial sequence
  primer
tcaccccagttgcaaattcacagcaatgaagtgct

SEQ ID NO: 181 - DNA, 35 bp, artificial sequence
  primer
tcaccccagttgcaaaattacagcaatgaagtgct

SEQ ID NO: 182 - DNA, 35 bp, artificial sequence
  primer
tcaccccagttgcaaactgacagcaatgaagtgct

SEQ ID NO: 183 - DNA, 35 bp, artificial sequence
  primer
tcaccccagttgcaaaccgacagcaatgaagtgct

SEQ ID NO: 184 - DNA, 35 bp, artificial sequence
  primer
tcaccccagttgcaaagtgacagcaatgaagtgct

SEQ ID NO: 185 - DNA, 35 bp, artificial sequence
  primer
tcaccccagttgcaaatggacagcaatgaagtgct

SEQ ID NO: 186 - DNA, 35 bp, artificial sequence
  primer
tcaccccagttgcaaaatgacagcaatgaagtgct

SEQ ID NO: 187 - DNA, 35 bp, artificial sequence
  primer
gaagtgctttctcttggcgttacaagttatttcac

SEQ ID NO: 188 - DNA, 35 bp, artificial sequence
  primer
gaagtgctttctcttgttcttacaagttatttcac

SEQ ID NO: 189 - DNA, 35 bp, artificial sequence
  primer
gaagtgctttctcttgattttacaagttatttcac

SEQ ID NO: 190 - DNA, 35 bp, artificial sequence
  primer
gaagtgctttctcttgctgttacaagttatttcac

SEQ ID NO: 191 - DNA, 35 bp, artificial sequence
  primer
gaagtgctttctcttgccgttacaagttatttcac

SEQ ID NO: 192 - DNA, 35 bp, artificial sequence
  primer
gaagtgctttctcttggtgttacaagttatttcac

SEQ ID NO: 193 - DNA, 35 bp, artificial sequence
  primer
gaagtgctttctcttgtggttacaagttatttcac

SEQ ID NO: 194 - DNA, 35 bp, artificial sequence
  primer
gaagtgctttctcttgatgttacaagttatttcac

SEQ ID NO: 195 - DNA, 35 bp, artificial sequence
  primer
ctgggtgaatgtaatagcggatttgaaaaaaattg

SEQ ID NO: 196 - DNA, 35 bp, artificial sequence
  primer
ctgggtgaatgtaatattcgatttgaaaaaaattg

SEQ ID NO: 197 - DNA, 35 bp, artificial sequence
  primer
ctgggtgaatgtaataattgatttgaaaaaaattg

SEQ ID NO: 198 - DNA, 35 bp, artificial sequence
  primer
ctgggtgaatgtaatactggatttgaaaaaaattg

SEQ ID NO: 199 - DNA, 35 bp, artificial sequence
  primer
ctgggtgaatgtaataccggatttgaaaaaaattg

SEQ ID NO: 200 - DNA, 35 bp, artificial sequence
  primer
ctgggtgaatgtaatagtggatttgaaaaaaattg

SEQ ID NO: 201 - DNA, 35 bp, artificial sequence
  primer
ctgggtgaatgtaatatgggatttgaaaaaaattg

SEQ ID NO: 202 - DNA, 35 bp, artificial sequence
  primer
ctgggtgaatgtaataatggatttgaaaaaaattg

SEQ ID NO: 203 - DNA, 35 bp, artificial sequence
  primer
tatgcatattgatgctgcgttatatacggaaagtg

SEQ ID NO: 204 - DNA, 35 bp, artificial sequence
  primer
tatgcatattgatgctttcttatatacggaaagtg

SEQ ID NO: 205 - DNA, 35 bp, artificial sequence
  primer
tatgcatattgatgctattttatatacggaaagtg

SEQ ID NO: 206 - DNA, 35 bp, artificial sequence
  primer
tatgcatattgatgctctgttatatacggaaagtg

SEQ ID NO: 207 - DNA, 35 bp, artificial sequence
  primer
tatgcatattgatgctccgttatatacggaaagtg

SEQ ID NO: 208 - DNA, 35 bp, artificial sequence
  primer
tatgcatattgatgctgtgttatatacggaaagtg

SEQ ID NO: 209 - DNA, 35 bp, artificial sequence
  primer
tatgcatattgatgcttggttatatacggaaagtg

SEQ ID NO: 210 - DNA, 35 bp, artificial sequence
  primer
tatgcatattgatgctatgttatatacggaaagtg

SEQ ID NO: 211 - DNA, 35 bp, artificial sequence
  primer
tacggaaagtgatgttgcgcccagttgcaaagtaa

SEQ ID NO: 212 - DNA, 35 bp, artificial sequence
  primer
tacggaaagtgatgttttccccagttgcaaagtaa

SEQ ID NO: 213 - DNA, 35 bp, artificial sequence
  primer
tacggaaagtgatgttattcccagttgcaaagtaa

SEQ ID NO: 214 - DNA, 35 bp, artificial sequence
  primer
tacggaaagtgatgttctgcccagttgcaaagtaa

SEQ ID NO: 215 - DNA, 35 bp, artificial sequence
  primer
tacggaaagtgatgttccgcccagttgcaaagtaa

SEQ ID NO: 216 - DNA, 35 bp, artificial sequence
  primer
tacggaaagtgatgttgtgcccagttgcaaagtaa

SEQ ID NO: 217 - DNA, 35 bp, artificial sequence
  primer
tacggaaagtgatgtttggcccagttgcaaagtaa

SEQ ID NO: 218 - DNA, 35 bp, artificial sequence
  primer
tacggaaagtgatgttatgcccagttgcaaagtaa

SEQ ID NO: 219 - DNA, 35 bp, artificial sequence
  primer
agtaacagcaatgaaggcgtttctcttggagttac

SEQ ID NO: 220 - DNA, 35 bp, artificial sequence
  primer
agtaacagcaatgaagttctttctcttggagttac

SEQ ID NO: 221 - DNA, 35 bp, artificial sequence
  primer
agtaacagcaatgaagatttttctcttggagttac

SEQ ID NO: 222 - DNA, 35 bp, artificial sequence
  primer
agtaacagcaatgaagctgtttctcttggagttac

SEQ ID NO: 223 - DNA, 35 bp, artificial sequence
  primer
agtaacagcaatgaagccgtttctcttggagttac

SEQ ID NO: 224 - DNA, 35 bp, artificial sequence
  primer
agtaacagcaatgaaggtgtttctcttggagttac

SEQ ID NO: 225 - DNA, 35 bp, artificial sequence
  primer
agtaacagcaatgaagtggtttctcttggagttac

SEQ ID NO: 226 - DNA, 35 bp, artificial sequence
  primer
agtaacagcaatgaagatgtttctcttggagttac

SEQ ID NO: 227 - DNA, 35 bp, artificial sequence
  primer
tctcttggagttacaagcgatttcacttgagtccg

SEQ ID NO: 228 - DNA, 35 bp, artificial sequence
  primer
tctcttggagttacaattcatttcacttgagtccg

SEQ ID NO: 229 - DNA, 35 bp, artificial sequence
  primer
tctcttggagttacaaattatttcacttgagtccg

SEQ ID NO: 230 - DNA, 35 bp, artificial sequence
  primer
tctcttggagttacaactgatttcacttgagtccg

SEQ ID NO: 231 - DNA, 35 bp, artificial sequence
  primer
tctcttggagttacaaccgatttcacttgagtccg

SEQ ID NO: 232 - DNA, 35 bp, artificial sequence
  primer
tctcttggagttacaagtgatttcacttgagtccg

SEQ ID NO: 233 - DNA, 35 bp, artificial sequence
  primer
tctcttggagttacaatggatttcacttgagtccg

SEQ ID NO: 234 - DNA, 35 bp, artificial sequence
  primer
tctcttggagttacaaatgatttcacttgagtccg

SEQ ID NO: 235 - DNA, 35 bp, artificial sequence
  primer
acttgagtccggagatgcgagtattcatgatacag

SEQ ID NO: 236 - DNA, 35 bp, artificial sequence
  primer
acttgagtccggagatttcagtattcatgatacag

SEQ ID NO: 237 - DNA, 35 bp, artificial sequence
  primer
acttgagtccggagatattagtattcatgatacag

SEQ ID NO: 238 - DNA, 35 bp, artificial sequence
  primer
acttgagtccggagatctgagtattcatgatacag

SEQ ID NO: 239 - DNA, 35 bp, artificial sequence
  primer
acttgagtccggagatccgagtattcatgatacag

SEQ ID NO: 240 - DNA, 35 bp, artificial sequence
  primer
acttgagtccggagatgtgagtattcatgatacag

SEQ ID NO: 241 - DNA, 35 bp, artificial sequence
  primer
acttgagtccggagattggagtattcatgatacag

SEQ ID NO: 242 - DNA, 35 bp, artificial sequence
  primer
acttgagtccggagatatgagtattcatgatacag

SEQ ID NO: 243 - DNA, 35 bp, artificial sequence
  primer
tattcatgatacagtagcgaatctgatcatcctag

SEQ ID NO: 244 - DNA, 35 bp, artificial sequence
  primer
tattcatgatacagtattcaatctgatcatcctag

SEQ ID NO: 245 - DNA, 35 bp, artificial sequence
  primer
tattcatgatacagtaattaatctgatcatcctag

SEQ ID NO: 246 - DNA, 35 bp, artificial sequence
  primer
tattcatgatacagtactgaatctgatcatcctag

SEQ ID NO: 247 - DNA, 35 bp, artificial sequence
  primer
tattcatgatacagtaccgaatctgatcatcctag

SEQ ID NO: 248 - DNA, 35 bp, artificial sequence
  primer
tattcatgatacagtagtgaatctgatcatcctag

SEQ ID NO: 249 - DNA, 35 bp, artificial sequence
  primer
tattcatgatacagtatggaatctgatcatcctag

SEQ ID NO: 250 - DNA, 35 bp, artificial sequence
  primer
tattcatgatacagtaatgaatctgatcatcctag

SEQ ID NO: 251 - DNA, 35 bp, artificial sequence
  primer
gatcatcctagcaaacgcgagtttgtcttctaatg

SEQ ID NO: 252 - DNA, 35 bp, artificial sequence
  primer
gatcatcctagcaaacttcagtttgtcttctaatg

SEQ ID NO: 253 - DNA, 35 bp, artificial sequence
  primer
gatcatcctagcaaacattagtttgtcttctaatg

SEQ ID NO: 254 - DNA, 35 bp, artificial sequence
  primer
gatcatcctagcaaacctgagtttgtcttctaatg

SEQ ID NO: 255 - DNA, 35 bp, artificial sequence
  primer
gatcatcctagcaaacccgagtttgtcttctaatg

SEQ ID NO: 256 - DNA, 35 bp, artificial sequence
  primer
gatcatcctagcaaacgtgagtttgtcttctaatg

SEQ ID NO: 257 - DNA, 35 bp, artificial sequence
  primer
gatcatcctagcaaactggagtttgtcttctaatg

SEQ ID NO: 258 - DNA, 35 bp, artificial sequence
  primer
gatcatcctagcaaacatgagtttgtcttctaatg

SEQ ID NO: 259 - DNA, 35 bp, artificial sequence
  primer
tttgtcttctaatggggcggtaacagaatctggat

SEQ ID NO: 260 - DNA, 35 bp, artificial sequence
  primer
tttgtcttctaatgggttcgtaacagaatctggat

SEQ ID NO: 261 - DNA, 35 bp, artificial sequence
  primer
tttgtcttctaatgggattgtaacagaatctggat

SEQ ID NO: 262 - DNA, 35 bp, artificial sequence
  primer
tttgtcttctaatgggctggtaacagaatctggat

SEQ ID NO: 263 - DNA, 35 bp, artificial sequence
  primer
tttgtcttctaatgggccggtaacagaatctggat

SEQ ID NO: 264 - DNA, 35 bp, artificial sequence
  primer
tttgtcttctaatggggtggtaacagaatctggat

SEQ ID NO: 265 - DNA, 35 bp, artificial sequence
  primer
tttgtcttctaatgggtgggtaacagaatctggat

SEQ ID NO: 266 - DNA, 35 bp, artificial sequence
  primer
tttgtcttctaatgggatggtaacagaatctggat

SEQ ID NO: 267 - DNA, 35 bp, artificial sequence
  primer
agaatctggatgcaaagcgtgtgaggaactggagg

SEQ ID NO: 268 - DNA, 35 bp, artificial sequence
  primer
agaatctggatgcaaattctgtgaggaactggagg

SEQ ID NO: 269 - DNA, 35 bp, artificial sequence
  primer
agaatctggatgcaaaatttgtgaggaactggagg

SEQ ID NO: 270 - DNA, 35 bp, artificial sequence
  primer
agaatctggatgcaaactgtgtgaggaactggagg

SEQ ID NO: 271 - DNA, 35 bp, artificial sequence
  primer
agaatctggatgcaaaccgtgtgaggaactggagg

SEQ ID NO: 272 - DNA, 35 bp, artificial sequence
  primer
agaatctggatgcaaagtgtgtgaggaactggagg

SEQ ID NO: 273 - DNA, 35 bp, artificial sequence
  primer
agaatctggatgcaaatggtgtgaggaactggagg

SEQ ID NO: 274 - DNA, 35 bp, artificial sequence
  primer
agaatctggatgcaaaatgtgtgaggaactggagg

SEQ ID NO: 275 - DNA, 35 bp, artificial sequence
  primer
atgtgaggaactggaggcgaaaaatattaaagaat

SEQ ID NO: 276 - DNA, 35 bp, artificial sequence
  primer
atgtgaggaactggagttcaaaaatattaaagaat

SEQ ID NO: 277 - DNA, 35 bp, artificial sequence
  primer
atgtgaggaactggagattaaaaatattaaagaat

SEQ ID NO: 278 - DNA, 35 bp, artificial sequence
  primer
atgtgaggaactggagctgaaaaatattaaagaat

SEQ ID NO: 279 - DNA, 35 bp, artificial sequence
  primer
atgtgaggaactggagccgaaaaatattaaagaat

SEQ ID NO: 280 - DNA, 35 bp, artificial sequence
  primer
atgtgaggaactggaggtgaaaaatattaaagaat

SEQ ID NO: 281 - DNA, 35 bp, artificial sequence
  primer
atgtgaggaactggagtggaaaaatattaaagaat

SEQ ID NO: 282 - DNA, 35 bp, artificial sequence
  primer
atgtgaggaactggagatgaaaaatattaaagaat

SEQ ID NO: 283 - DNA, 35 bp, artificial sequence
  primer
tattaaagaatttttggcgagttttgtacatattg

SEQ ID NO: 284 - DNA, 35 bp, artificial sequence
  primer
tattaaagaatttttgttcagttttgtacatattg

SEQ ID NO: 285 - DNA, 35 bp, artificial sequence
  primer
tattaaagaatttttgattagttttgtacatattg

SEQ ID NO: 286 - DNA, 35 bp, artificial sequence
  primer
tattaaagaatttttgctgagttttgtacatattg

SEQ ID NO: 287 - DNA, 35 bp, artificial sequence
  primer
tattaaagaatttttgccgagttttgtacatattg

SEQ ID NO: 288 - DNA, 35 bp, artificial sequence
  primer
tattaaagaatttttggtgagttttgtacatattg

SEQ ID NO: 289 - DNA, 35 bp, artificial sequence
  primer
tattaaagaatttttgtggagttttgtacatattg

SEQ ID NO: 290 - DNA, 35 bp, artificial sequence
  primer
tattaaagaatttttgatgagttttgtacatattg

SEQ ID NO: 291 - DNA, 35 bp, artificial sequence
  primer
ttttgtacatattgtcgcgatgttcatcaacactt

SEQ ID NO: 292 - DNA, 35 bp, artificial sequence
  primer
ttttgtacatattgtcttcatgttcatcaacactt

SEQ ID NO: 293 - DNA, 35 bp, artificial sequence
  primer
ttttgtacatattgtcattatgttcatcaacactt

SEQ ID NO: 294 - DNA, 35 bp, artificial sequence
  primer
ttttgtacatattgtcctgatgttcatcaacactt

SEQ ID NO: 295 - DNA, 35 bp, artificial sequence
  primer
ttttgtacatattgtcccgatgttcatcaacactt

SEQ ID NO: 296 - DNA, 35 bp, artificial sequence
  primer
ttttgtacatattgtcgtgatgttcatcaacactt

SEQ ID NO: 297 - DNA, 35 bp, artificial sequence
  primer
ttttgtacatattgtctggatgttcatcaacactt

SEQ ID NO: 298 - DNA, 35 bp, artificial sequence
  primer
ttttgtacatattgtcatgatgttcatcaacactt

SEQ ID NO: 299 - DNA, 767 bp, artificial sequence
  Clone 1
aggagggccaccatgcgaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttttccatattgtccaaatgttcatcaacacttcttgattgcaattgagctagcattatccctaatacctgccaccccactcttaatcagtggtggaagaacggtctcagaactgtttgtttcaattggccatttaagtttagtagtaaaagactggttaatgataacaatgcatcgtaaaagctttcagaaggaaaggagaatgttttgtgatctactttggttttcttttttgcgtgtggcagttttaagttattagtttttaaaatcagtactttttaatggaaacaacttgaccaaaaat

SEQ ID NO: 300 - DNA, 767 bp, artificial sequence
  clone 2
aggagggccaccatgcgaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagcgcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgattgcaattgagctagcattatccctaatacctgccaccccactcttaatcagtggtggaagaacggtctcagaactgtttgtttcaattggccatttaagtttagtagtaaaagactggttaatgataacaatgcatcgtaaaagctttcagaaggaaaggagaatgttttgtgatctactttggttttcttttttgcgtgtggcagttttaagttattagtttttaaaatcagtactttttaatggaaacaacttgaccaaaaat

SEQ ID NO: 301 - DNA, 767 bp, artificial sequence
  clone 3
aggagggccaccatgcgaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttccgcatattgtccaaatgttcatcaacacttcttgattgcaattgagctagcattatccctaatacctgccaccccactcttaatcagtggtggaagaacggtctcagaactgtttgtttcaattggccatttaagtttagtagtaaaagactggttaatgataacaatgcatcgtaaaagctttcagaaggaaaggagaatgttttgtgatctactttggttttcttttttgcgtgtggcagttttaagttattagtttttaaaatcagtactttttaatggaaacaacttgaccaaaaat

SEQ ID NO: 302 - DNA, 767 bp, artificial sequence
  clone 4
aggagggccaccatgcgaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtaccgattgtccaaatgttcatcaacacttcttgattgcaattgagctagcattatccctaatacctgccaccccactcttaatcagtggtggaagaacggtctcagaactgtttgtttcaattggccatttaagtttagtagtaaaagactggttaatgataacaatgcatcgtaaaagctttcagaaggaaaggagaatgttttgtgatctactttggttttcttttttgcgtgtggcagttttaagttattagtttttaaaatcagtactttttaatggaaacaacttgaccaaaaat

SEQ ID NO: 303 - DNA, 767 bp, artificial sequence; identical to SEQ ID NO: 302
  clone 5

SEQ ID NO: 304 - DNA, 767 bp, artificial sequence
  clone 6
aggagggccaccatgcgaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttttccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacatggtctggatgcaaagaatgtgaggaactggaggaaaaaaatattaaagaatttttgcagagttttgtaccgattgtccaaatgttcatcaacacttcttgattgcaattgagctagcattatccctaatacctgccaccccactcttaatcagtggtggaagaacggtctcagaactgtttgtttcaattggccatttaagtttagtagtaaaagactggttaatgataacaatgcatcgtaaaagctttcagaaggaaaggagaatgttttgtgatctactttggttttcttttttgcgtgtggcagttttaagttattagtttttaaaatcagtactttttaatggaaacaacttgaccaaaaat

SEQ ID NO: 305 - DNA, 767 bp, artificial sequence
  clone 7
aggagggccaccatgcgaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgattgcaattgagctagcattatccctaatacctgccaccccactcttaatcagtggtggaagaacggtctcagaactgtttgtttcaattggccatttaagtttagtagtaaaagactggttaatgataacaatgcatcgtaaaagctttcagaaggaaaggagaatgttttgtgatctactttggttttcttttttgcgtgtggcagttttaagttattagtttttaaaatcagtactttttaatggaaacaacttgaccaaaaat

SEQ ID NO: 306 - DNA, 767 bp, artificial sequence; identical to SEQ ID NO: 305
  clone 8

SEQ ID NO: 307 - DNA, 767 bp, artificial sequence; identical to SEQ ID NO: 302
  clone 9

SEQ ID NO: 308 - DNA, 767 bp, artificial sequence
  clone 10
aggagggccaccatgcgaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaactgcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgattgcaattgagctagcattatccctaatacctgccaccccactcttaatcagtggtggaagaacggtctcagaactgtttgtttcaattggccatttaagtttagtagtaaaagactggttaatgataacaatgcatcgtaaaagctttcagaaggaaaggagaatgttttgtgatctactttggttttcttttttgcgtgtggcagttttaagttattagtttttaaaatcagtactttttaatggaaacaacttgaccaaaaat

SEQ ID NO: 309 - DNA, 767 bp, artificial sequence; identical to SEQ ID NO: 300
  clone 11

SEQ ID NO: 310 - DNA, 767 bp, artificial sequence; identical to SEQ ID NO: 305
  clone 12

SEQ ID NO: 311 - DNA, 767 bp, artificial sequence
  clone 13
aggagggccaccatgcgaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacgatgagtgatgttcaccccagttgcaaagtaacagtgatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtaccgattgtccaaatgttcatcaacacttcttgattgcaattgagctagcattatccctaatacctgccaccccactcttaatcagtggtggaagaacggtctcagaactgtttgtttcaattggccatttaagtttagtagtaaaagactggttaatgataacaatgcatcgtaaaagctttcagaaggaaaggagaatgttttgtgatctactttggttttcttttttgcgtgtggcagttttaagttattagtttttaaaatcagtactttttaatggaaacaacttgaccaaaaat

SEQ ID NO: 312 - DNA, 767 bp, artificial sequence
  clone 14
aggagggccaccatgcgaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaaatgcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgattgcaattgagctagcattatccctaatacctgccaccccactcttaatcagtggtggaagaacggtctcagaactgtttgtttcaattggccatttaagtttagtagtaaaagactggttaatgataacaatgcatcgtaaaagctttcagaaggaaaggagaatgttttgtgatctactttggttttcttttttgcgtgtggcagttttaagttattagtttttaaaatcagtactttttaatggaaacaacttgaccaaaaat

SEQ ID NO: 313 - DNA, 767 bp, artificial sequence
  clone 15
aggagggccaccatgcgaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgtgtccggagatgcaagtattattgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgattgcaattgagctagcattatccctaatacctgccaccccactcttaatcagtggtggaagaacggtctcagaactgtttgtttcaattggccatttaagtttagtagtaaaagactggttaatgataacaatgcatcgtaaaagctttcagaaggaaaggagaatgttttgtgatctactttggttttcttttttgcgtgtggcagttttaagttattagtttttaaaatcagtactttttaatggaaacaacttgaccaaaaat

SEQ ID NO: 314 - DNA, 767 bp, artificial sequence
  clone 16
aggagggccaccatgcgaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaatggattaaagaatttttgcagagttttgtacatattgtccaaatgttcatcaacacttcttgattgcaattgagctagcattatccctaatacctgccaccccactcttaatcagtggtggaagaacggtctcagaactgtttgtttcaattggccatttaagtttagtagtaaaagactggttaatgataacaatgcatcgtaaaagctttcagaaggaaaggagaatgttttgtgatctactttggttttcttttttgcgtgtggcagttttaagttattagtttttaaaatcagtactttttaatggaaacaacttgaccaaaaat

SEQ ID NO: 315 - DNA, 767 bp, artificial sequence
  clone 17
aggagggccaccatgcgaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaattattaaagaatttttgcagagttttgtacatattgtccaaatgttcatcaacacttcttgattgcaattgagctagcattatccctaatacctgccaccccactcttaatcagtggtggaagaacggtctcagaactgtttgtttcaattggccatttaagtttagtagtaaaagactggttaatgataacaatgcatcgtaaaagctttcagaaggaaaggagaatgttttgtgatctactttggttttcttttttgcgtgtggcagttttaagttattagtttttaaaatcagtactttttaatggaaacaacttgaccaaaaat

SEQ ID NO: 316 - DNA, 767 bp, artificial sequence; identical to SEQ ID NO: 315
  clone 18

SEQ ID NO: 317 - DNA, 767 bp, artificial sequence
  clone 19
aggagggccaccatgcgaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctggcaaacaacagtttgtcttctaatgggaatgtaacaccgtctggatgcaaagaatgtgaggaactggaggaaaaactgattaaagaatttttgcagagttttgtacatattgtccaaatgttcatcaacacttcttgattgcaattgagctagcattatccctaatacctgccaccccactcttaatcagtggtggaagaacggtctcagaactgtttgtttcaattggccatttaagtttagtagtaaaagactggttaatgataacaatgcatcgtaaaagctttcagaaggaaaggagaatgttttgtgatctactttggttttcttttttgcgtgtggcagttttaagttattagtttttaaaatcagtactttttaatggaaacaacttgaccaaaaat

SEQ ID NO: 318 - DNA, 767 bp, artificial sequence; identical to SEQ ID NO: 308
  clone 20

SEQ ID NO: 319 - DNA, 767 bp, artificial sequence
  clone 21
aggagggccaccatgcgaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagtgcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgattgcaattgagctagcattatccctaatacctgccaccccactcttaatcagtggtggaagaacggtctcagaactgtttgtttcaattggccatttaagtttagtagtaaaagactggttaatgataacaatgcatcgtaaaagctttcagaaggaaaggagaatgttttgtgatctactttggttttcttttttgcgtgtggcagttttaagttattagtttttaaaatcagtactttttaatggaaacaacttgaccaaaaat

SEQ ID NO: 320 - DNA, 767 bp, artificial sequence
  clone 22
aggagggccaccatgcgaatttcgaaaccacatttgagaagtatttccatccagtgctacttgtgtttacttctaaacagtcattttctaactgaatggggcattcatgtcttcattttgggctgtttcagtgcagggcttcctaaaacagaagccaactgggtgaatgtaataagtgatttgaaaaaaattgaaattcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgattgcaattgagctagcattatccctaatacctgccaccccactcttaatcagtggtggaagaacggtctcagaactgtttgtttcaattggccatttaagtttagtagtaaaagactggttaatgataacaatgcatcgtaaaagctttcagaaggaaaggagaatgttttgtgatctactttggttttcttttttgcgtgtggcagttttaagttattagtttttaaaatcagtactttttaatggaaacaacttgaccaaaaat

SEQ ID NO: 321 - DNA, 767 bp, artificial sequence
  clone 23
aggagggccaccatgcgaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacatggtctggatgcaaagaatgtgaggaactggaggaaaaaaatattaaagaatttttgcagagttttgtaccgattgtccaaatgttcatcaacacttcttgattgcaattgagctagcattatccctaatacctgccaccccactcttaatcagtggtggaagaacggtctcagaactgtttgtttcaattggccatttaagtttagtagtaaaagactggttaatgataacaatgcatcgtaaaagctttcagaaggaaaggagaatgttttgtgatctactttggttttcttttttgcgtgtggcagttttaagttattagtttttaaaatcagtactttttaatggaaacaacttgaccaaaaat

SEQ ID NO: 322 - DNA, 767 bp, artificial sequence
  clone 24
aggagggccaccatgcgaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccgcgtgttcatcaacacttcttgattgcaattgagctagcattatccctaatacctgccaccccactcttaatcagtggtggaagaacggtctcagaactgtttgtttcaattggccatttaagtttagtagtaaaagactggttaatgataacaatgcatcgtaaaagctttcagaaggaaaggagaatgttttgtgatctactttggttttcttttttgcgtgtggcagttttaagttattagtttttaaaatcagtactttttaatggaaacaacttgaccaaaaat

SEQ ID NO: 323 - DNA, 767 bp, artificial sequence
  clone 25
aggagggccaccatgcgaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattattgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgattgcaattgagctagcattatccctaatacctgccaccccactcttaatcagtggtggaagaacggtctcagaactgtttgtttcaattggccatttaagtttagtagtaaaagactggttaatgataacaatgcatcgtaaaagctttcagaaggaaaggagaatgttttgtgatctactttggttttcttttttgcgtgtggcagttttaagttattagtttttaaaatcagtactttttaatggaaacaacttgaccaaaaat

SEQ ID NO: 324 - DNA, 767 bp, artificial sequence
  clone 26
aggagggccaccatgcgaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttgttcttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgattgcaattgagctagcattatccctaatacctgccaccccactcttaatcagtggtggaagaacggtctcagaactgtttgtttcaattggccatttaagtttagtagtaaaagactggttaatgataacaatgcatcgtaaaagctttcagaaggaaaggagaatgttttgtgatctactttggttttcttttttgcgtgtggcagttttaagttattagtttttaaaatcagtactttttaatggaaacaacttgaccaaaaat

SEQ ID NO: 325 - DNA, 767 bp, artificial sequence
  clone 27
aggagggccaccatgcgaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctggcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgattgcaattgagctagcattatccctaatacctgccaccccactcttaatcagtggtggaagaacggtctcagaactgtttgtttcaattggccatttaagtttagtagtaaaagactggttaatgataacaatgcatcgtaaaagctttcagaaggaaaggagaatgttttgtgatctactttggttttcttttttgcgtgtggcagttttaagttattagtttttaaaatcagtactttttaatggaaacaacttgaccaaaaat

(19) United States
(12) Patent Application Publication
(10) Pub. No.: US 2005/0153343 A1
(43) Pub. Date: Jul. 14, 2005

(54) Method of massive directed mutagenesis

(75) Inventors: Julien Sylvestre, Paris (FR); Marc Delcourt, Paris (FR)

Correspondence Address: NIXON & VANDERHYE, PC, 1100 N GLEBE ROAD, 8TH FLOOR, ARLINGTON, VA, 22201-4714, US

(73) Assignee: BIOMETHODES, Evry (FR)

(21) Appl. No.: 11/012,068

(22) Filed: Dec. 15, 2004

(30) Foreign Application Priority Data
Dec. 18, 2003 (FR) FR 03 14892

(51) Int. Cl. C12Q 1/68; C12N 15/85

(52) U.S. Cl. 435/6; 435/455

(57) ABSTRACT

The invention relates to the field of molecular biology and more particularly that of mutagenesis. The invention has as its object a method of high throughput directed mutagenesis, that is to say, constitution of a large number of directed mutants at a reduced cost, time and number of steps. The invention also relates to the double stranded polynucleotides so obtained and the peptides, polypeptides, or proteins so obtained having one or more improved properties, and the uses of said method.

### FIELD OF THE INVENTION

The invention relates to the field of molecular biology and more particularly that of mutagenesis. The invention has as its object a method of high throughput directed mutagenesis, that is to say, constitution of a large number of directed mutants at a reduced time, cost and number of steps. The invention also relates to the double stranded polynucleotides so obtained and the peptides, polypeptides, or proteins so obtained having one or more improved properties, and the uses of said method.

### BACKGROUND OF THE INVENTION

Mutagenesis is a technique that aims to artificially modify the nucleotide sequence of a DNA fragment, with the intention of modifying the biological activity resulting therefrom.

The term mutagenesis can be associated with three distinct modifications of a DNA fragment:
deletion, which corresponds to removal of one or more nucleotides from the DNA fragment of interest;
insertion, which corresponds to addition of same;
substitution, which corresponds to replacement of one or more bases with a same number of bases of different nature.

Mutagenesis plays a key role in the field of protein improvement, and principally of therapeutic proteins and enzymes.

Enzyme improvement has a major economic interest: indeed, a great number of industrial enzymes are used in various processes—such as vitamin or antibiotic synthesis, beer production, textile treatments—or in products as diverse as detergents and cattle feed (Turner et al., Trends Biotechnol. 2003 Nov. 21(11): 474-8). Improvement of enzymes makes it possible to lower the costs of the corresponding processes, or to implement new processes.

The parameters to be improved are varied. For example, and not by way of limitation, by molecular evolution it is possible to obtain an enzyme with an extremely high turnover (Griffiths A D et al., EMBO J. 2003, 22(1): 24-35); obtain an enzyme with increased thermostability (Baik S H et al., Appl. Microbiol. Biotechnol. 2003, 61(4): 329-35); optimize a therapeutic protein (Vasserot A P et al., Drug Discov. Today 2003, 8(3): 118-26); obtain a peptide which binds with high affinity to a given ligand (Lamla T et al., J. Mol. Biol. 2003, 329(2): 381-8); create in vitro an antibody against virtually any ligand, for use in diagnostics (Azzazy H M et al., Clin. Biochem. 2002, 35(6): 425-45); create a ribozyme with a novel catalytic activity (McGinness K E et al., Chem. Biol. 2002, 9(5): 585-96; Sun L. et al., Chem. Biol. 2002, 9(5): 619-28). The parameters to be improved may be multiple: for example, by molecular evolution it is possible to obtain an enzyme resistant to both heat and oxidation (Oh K H. et al., Protein Eng. 2002, 15(8): 689-95) or to broaden the pH range in which the enzyme is effective all while increasing the activity thereof (Bessler C. et al., Protein Sci. 2003 Oct. 12(10): 2141-9). Finally, today enzymes make it possible to replace certain heavy and polluting chemical methods with methods that are far more environmentally friendly (so-called green chemistry).

In the field of therapeutic proteins, the production of mutant proteins having novel properties also has a major therapeutic and economic interest: the isolation of mutant EPO having a longer half-life or of long-acting insulin are examples of successful production of new generations of therapeutic proteins by mutagenesis. Among the therapeutic proteins for which improvements might be interesting, particular examples include hormones, cytokines, interferons, vaccines and antibodies.

In this context of seeking mutants having acquired a novel property or having an improved existing property, mutagenesis constitutes a first step and creates diversity. In a second step, said diversity is then screened by means of a functional test, so as to isolate a mutant molecule coding for an improved protein. Generally this is a rare event, and a large number of mutant molecules must be analyzed before obtaining an improved molecule.

Different approaches to mutagenesis may be implemented in this context:

A rational approach which is based on the use of a physiochemical rationale and/or structural data and/or bioinformatics modelling to generate a small set of hypotheses, for which a small number of corresponding mutants will be generated. It can be predicted that these few mutants will each have a high probability of corresponding to an improved protein. Quite often, however, the relative scarcity of protein crystallographic data and the poor quality of bioinformatics-based predictions make this approach risky.

A “molecular evolution” approach which is supposed to imitate the natural evolution of genes in an accelerated manner and in vitro. Large numbers of variants are randomly generated. These mutants are then screened individually (high throughput screening) or in bulk (selection methods). In most cases, the number of mutants to be screened is extremely high (typically: 10⁶-10¹²) since the very large majority of mutants are not improved and since a large library is needed to investigate a reasonably interesting sequence space. In the absence of a mass selection system, this approach often proves to be tedious.

Between these two extremes, mixed strategies can exist: a large number of mutants, some randomly generated and some rationally based, can be designed and produced. In this case it is expected that the frequency of improved mutants in these semirational libraries will be higher than if diversity were generated solely on a random basis; screening requirements would therefore be reduced.

The applications of molecular evolution are not limited to the discovery of proteins with novel or improved properties. Evolution of nucleic acids in the laboratory is also possible. In addition to their value in fundamental research (Mc Giness K E et al., Chem. Biol. 2003 Jan. 10(1): 5-14), some of these RNAs and DNAs (particularly ribozymes) can be of interest in biotechnology, diagnostics or therapeutics. Approaches using long degenerate oligonucleotides or random mutagenesis have produced promising early results, particularly in the field of “continuous evolution” (Mc Ginness K E et al., Chem. Biol. 2002 May 9(5): 585-96; Tsukiji S., Nat. Struct. Biol. 2003 Sep. 10(9): 713-7; Ricca B I et al., J. Mol. Biol. 2003 Jul. 25; 330(5): 1015-25; Khan A U et al., J. Biomed. Sci. 2003 Sep.-Oct. 10(5): 457-67). A specific, high-throughput method by which to evolve (mutate) and then select one or more molecules of this type would potentially be complementary.

Mutagenesis also has an interest and an opposite use: to create mutations associated with a reduction of biological activity. This approach is usually part of upstream research on protein structure/function relationships, and more particularly is aimed at identifying the residues directly involved in the activity of the protein under study. Said approach is not usually associated with an immediate industrial application.

If modification of an amino acid results in loss of biological activity, it is likely that this amino acid plays a role in formation of the active site underlying said biological activity. However, this conclusion should be viewed with a great deal of caution, because alternatively it is possible that said amino acid is not directly involved in the active site underlying the biological activity, but rather in associated activities (like intracellular signalling of the protein for example), or else that the modification introduced thereto destabilizes the protein as a whole, in which case the effect of the substitution would be indirect, and not direct.

It is then important to know how to recognize the motifs underlying the activities of signalling, membrane localization, cofactor binding, and the like.

Moreover, it is essential that the modifications introduced cause the least possible destabilization of the protein secondary structure. This is why, most of the time, the original amino acids are substituted by an alanine. It is known that this small amino acid mostly preserves secondary structure of proteins (alpha helix and beta sheet), does not induce major steric or electrical alterations, and therefore keeps global protein destabilization to a minimum.

Studies in this field require the generation of a large number of point mutants each comprising an alanine substitution of an amino acid. Said mutants must then be studied individually by means of functional tests, to evaluate the effect of the substitution introduced. Several hundred articles based on this method have been published. The principle of alanine scanning, and the merits and limitations of this strategy are discussed in particular in the review of DeLano W L. (Curr. Opin. Struct. Biol. 2002 Feb. 12(1): 14-20) and Morrisson K L. et al. (Curr. Opin. Chem. Biol. 2001 Jun. 5(3): 302-7); the ASEdb data base centralizes many alanine scan results (Thorn K S et al. Bioinformatics 2001 Mar. 17(3): 284-5). In some cases, the amino acid residues are systematically substituted by cysteines and not by alanines (Tamura N et al., Curr. Opin. Chem. Biol. 2003 Oct. 7(5): 570-9; Winkler H H et al., Biochemistry 2003 Nov. 4; 42(43): 12562-9). More generally, any type of systematic substitution by a given amino acid can be envisioned. In the same perspective of using molecular evolution not directly to improve proteins, but for research purposes to generate data by which to analyze protein structure-function relationships, Christ D. et al. recently described an approach based on semi-random mutagenesis (Proc. Natl. Acad. Sci. USA 2003 Oct. 22).

In summary, mutagenesis is a tool allowing to obtain improved molecules having an economic interest more particularly in the field of biocatalysis (industrial enzymes) and medicine (therapeutic proteins). Mutagenesis is also an approach allowing to characterize proteins for research purposes, by identifying the amino acids in a protein that are directly related to the function thereof.

Although the main economic value lies in the protein field, mutagenesis and molecular evolution of DNA and RNA, in particular of RNA with catalytic properties (ribozymes), can be of interest. High throughput site-directed mutagenesis is also interesting in this context.

Various mutagenesis methods have been developed over the past few decades, and can be used in these different contexts.

Mutagenesis methods can be divided into five main groups:
random mutagenesis;
mutagenesis by DNA shuffling (recombination);
directed mutagenesis;
saturation mutagenesis;
semi-random mutagenesis.

Random mutagenesis aims to introduce substitutions of uncontrolled nature and position into a DNA fragment.

Historically, random mutagenesis was carried out by chemical methods altering the DNA structure (Richie D A. Genet Res. 1965 Nov. 6(3): 474-8 and Bridges B A. Mutat. Res. 1966 Aug. 3(4): 273-9).

A second approach to generate random mutants is to transform a plasmid containing the gene of interest into so-called “mutator” bacterial strains (Giraud A et al., Curr. Opin. Microbiol. 2001 Oct. 4(5): 582-5), which are deficient in some of the genes involved in fidelity of DNA replication (Irving R A et al., Methods Mol. Biol. 2002; 178: 295-302). Said approach is rarely used today, in particular due to problems linked to the genetic instability of this type of strain.

More recently, a great number of documents have described random mutagenesis methods based either on the use of a modified polymerase having a structurally low fidelity of replication, or on the use of a non-modified polymerase, but under specific amplification conditions leading to a high mutation rate (mutagenic PCR or ‘error-prone PCR’ is reviewed in Cirino P C et al., Methods Mol. Biol. 2003; 231: 3-9; Leung, D. W. et al., (1989) Technique 1: 11-15; Cadwell, R. C. and Joyce, G. F. (1992) PCR Methods Appl. 2: 28-33.). In both cases, the enzyme introduces mutations at each round; at the end of the reaction, many copies of the starting molecule are obtained, each molecule bearing one or more different mutations. Said molecules are present in the form of a library, that is, a mixture of molecules of different nature. The average number of mutations per molecule can be controlled by adjusting the different parameters of the mutagenesis reaction.

Random mutagenesis has considerable limitations. For instance, the mutations introduced by the polymerase usually do not concern several contiguous nucleotides, but just one. Using the random mutagenesis approach, only some of the 64 possible codons can be obtained from these single substitutions and, on average, only 5 of the 19 possible amino acids can be obtained from the starting codon. Moreover, each base is not substituted by each of the other bases with equal probability, which introduces bias into the DNA populations created as compared with ideal populations where any A, for example, would have the same probability of being substituted by a T, a C or a G.

In addition, one of the limitations of random mutagenesis stems from the need to clone the DNA fragment obtained in the mutagenic PCR reaction into a linearized vector. This cloning step often turns out to be the limiting factor when one seeks to obtain a large number of mutant molecules. In fact, the ligation step limits the size of the library to about 10⁶.

Mutagenesis by DNA shuffling takes its inspiration from the recombination process at work in Darwinian evolution, in particular during sexual reproduction. Mutagenesis by DNA shuffling consists of recombining partially homologous sequences, isolated from different organisms. For example, if one is working on an enzyme, the first step in a DNA shuffling approach will be to isolate a large number of genes homologous to said enzyme, either from collections of strains, or from genes directly isolated from natural samples (by what is now described as a “metagenome” approach). Different approaches are then available by which to shuffle the domains of these homologous genes and generate a library of “chimeric” DNA molecules, that is, composed of several domains from different sources [(Maxygen patent U.S. Pat. No. 6,132,970; Stemmer W P et al., Nature 1994 Aug. 4; 370(6488): 389-91; Aguinaldo A M et al., Methods Mol. Biol. 2002; 192: 235-9; Zhao H et al., Nat. Biotechnol. 1998 Mar. 16(3): 258-61; Shao, Z et al., Nucleic Acids Res. 26 (2): 681-683; Kawarasaki Y et al., Nucleic Acids Res. 2003 Nov. 1; 31(21): e126; Diversa patent U.S. Pat. No. 5,965,408; Proteus patent WO 00 09 679; Alligator patent WO 02 48 351). It is expected that said molecules thus contain novel characteristics, and in particular the combined properties of two or more parental genes. So, for example, starting with two genes homologous to a same enzyme, one known to be highly active, and the other to be thermostable (the latter having been isolated for example from a thermophilic organism), it might be hoped that some of the molecules obtained by shuffling said two genes—so containing some domains of the first and other domains of the second—will have the combined properties of high activity and thermostability (such additivity is not a given and does not always occur but in practice is quite frequently observed). In some cases, not only combined properties but also novel properties (for example an activity superior to that of the two natural parental genes) have been obtained by gene shuffling.

This DNA recombination approach is based on the general idea that the novel combination of natural mutations—which have therefore been prescreened by nature to maintain the activity of the enzyme—has a greater probability of conferring an improvement than introduction of randomly generated mutations. However, at the same time that one restricts the diversity to a sequence space which is “reasonable” because it is “preselected”, one is nonetheless limited by the original sequences, which must be known, by the need to have genes sharing a sufficient level of homology, and by the impossibility of generating sequences other than combinations of the original sequences.

Said DNA shuffling approaches have proved to be particularly efficient in the field of enzyme improvement. On the other hand, said approach is not adapted to the field of therapeutic proteins, on the one hand because human polymorphisms are fairly limited, and on the other hand because it is hardly conceivable, for reasons of immunogenicity in particular, to think that shuffling DNA from proteins of different species can provide a notable benefit in human therapeutics.

Directed mutagenesis aims to introduce one or more mutations the nature and position of which are known, into a recombinant gene. Said mutation or mutations are introduced by means of an oligonucleotide. Said oligonucleotide is classically composed of twenty to thirty bases homologous to the targeted region and at whose center are located the desired mutation(s).

Said oligonucleotide is used to prime a replication reaction (or an amplification, i.e., multiple replications) by using the DNA fragment as template. The newly synthesized sequence then contains the desired modification.

The first directed mutagenesis methods were based on amplification of a linear DNA fragment, when then had to be cloned into a plasmid using restriction enzymes.

More recently, the mutant oligonucleotide has been used to prime circular replication of the plasmid containing the DNA fragment of interest. This minimizes the number of needed manipulations. However, a selection step is necessary to separate molecules having effectively integrated the mutation from the starting DNA molecules. Said mutant selection step can be based on the use of specific organisms, such as the ung− bacterial strain (Kurikel T A, Bebenek K, McClary J. Methods Enzymol. 1991; 204: 125-39.), or phage M13. It can also be based on the simultaneous introduction of a second mutation which cosegregates with the first and which is selectable by a criterion of antibiotic resistance (EP 0938552), or by modification of a unique restriction site (Clontech Catalog 2000, page 45).

These approaches are now obsolete, and today the most widely used approach is based on differences in methylation between DNA synthesized in bacteria (methylated) and DNA synthesized in vitro (not methylated). A screening system based on this criterion was developed and is now widespread: it makes use of the enzyme DpnI, specific for sites present on methylated DNA but not on unmethylated DNA (Lacks et al., 1980, Methods in Enzymology, 65: 138). The enzymes NanII, NmuDI and NmuEI can also be used for the same purpose. In a mutagenesis reaction by circular elongation of an oligonucleotide hybridized to a plasmid, said enzymes digest the parental strands (which are produced in vivo by the bacteria and methylated), but not the unmethylated strands synthesized in the mutagenesis reaction. Digestion with said enzymes therefore results in an increase in the frequency of mutant molecules by eliminating non-mutant parental strands.

The effect of said enzymes on molecular species in which both strands are identical is clear, but their action on DNA molecules in which only one of the two strands is methylated—the other having been de novo synthesized—has not been as clearly established. Nonetheless, it is likely that said molecules, sometimes called “heteroduplexes”, cannot be efficiently digested by the enzymes DpnI, NanII, NmuDI or NmuEI. Now, when a single mutant oligonucleotide is used, the heteroduplexes supposedly constitute the majority (the desired mutation is only present on one of the two strands). After digestion with one of the above enzymes, a high mutagenesis rate would be expected (50%). Yet this efficiency remains low and mutant molecules usually comprise between 1 and 10%, as the case may be. This low mutant frequency is due in particular to the fact that at the end of the mutagenesis reaction, the heteroduplexes are introduced into bacteria containing a DNA repair system, which uses the methylated (and therefore non-mutant) strand as template to be copied. This repair system therefore results in repair of the introduced mutation, and a significant loss in mutagenesis yield.

To improve mutagenesis activity, the use of a second oligonucleotide, intended for synthesis of the second strand, is recommended.

Said second oligonucleotide can be non-mutant, and located in a region different from the region to be mutated (EP 96942905; WO 9935281).

Alternatively, the second oligonucleotide can have reverse complementarity to the first, and so contain the mutation as well. It is this latter approach which gives the best mutagenesis yields and which forms the basis of the Quickchange method (Stratagene 2003 reference #200518). Said method has become the standard for mutagenesis in recent years, due to its unequalled yield.

Directed mutagenesis is a very powerful technological approach. Its main limitation is throughput: indeed, it is scarcely possible to generate more than one or a few directed mutants per day and per person.

The fourth approach by which to generate diversity is “saturation mutagenesis”. Said approach consists of using oligonucleotides to generate in a target codon not a single substitution but a set of mutants containing the 64 possible codons, or a subset of these 64 codons.

Saturation mutagenesis is based on the use of degenerate oligonucleotides. During oligonucleotide synthesis, it is easy to create degeneration at any site in the oligonucleotide sequence by using, at the desired position, not a single base but an equimolar mixture of several bases. In a sequence, N conventionally denotes the equimolar mixture of the four bases. For example, ATN corresponds to 25% ATA, 25% ATT, 25% ATG and 25% ATC. An oligonucleotide containing two degenerate positions is in fact composed of an equimolar mixture of 16 oligonucleotides. An oligonucleotide with three degenerate positions N is composed of an equimolar mixture of 64 oligonucleotides. Said oligonucleotides containing degenerate bases are generally available at no extra cost from companies specializing in oligonucleotide synthesis.

Oligonucleotides containing a totally degenerate codon (NNN) make it possible to introduce maximum diversity, i.e., the 64 possible codons, at a site. From these 64 possible codons, the 19 possible amino acid substitutions will be translated.

Introduction of degenerate oligonucleotides can be done by using virtually any directed mutagenesis method, although it has been observed that the Quickchange method is not well suited to this approach.

In comparison with directed mutagenesis, saturation mutagenesis considerably reduces the work needed to produce a mutant molecule: several saturation mutants can be generated per day by a single person, which corresponds in each case to 19 different mutants. Nonetheless, this increased efficiency is only achieved at the cost of certain technical concessions:

Each of the 19 amino acids is integrated at different frequencies due to the degeneration of the genetic code. For instance, serine is integrated six times more often than tryptophane, and three times more often than aspartic acid. It would therefore require an enormous effort to isolate mutants corresponding to amino acids represented only once or twice.

In 3 cases out of 64, a stop codon is integrated, meaning that approximately 5% (3/64) of clones produced are simply of no interest.

A large portion of the codons introduced corresponds to codons which are minimally represented in the organism used to express the mutant molecules. Said codons with low representation are generally unfavorable to expression, and can complicate the analysis by masking a positive mutation.

One solution to keep these shortcomings to a minimum is to use partially degenerate oligonucleotides, that is, composed of codons of the type NNG/T (also called NNK) at the codon to be modified. Several solutions have been proposed for large scale saturation mutagenesis: Savino et al, PNAS 1993 90: 4067-71; Olins et al., Journal of Biological Chemistry 1995 270: 23754-60; patents U.S. Pat. No. 6,562,594 and U.S. Pat. No. 6,171,820 (Diversa); U.S. Pat. No. 6,180,341 (University of Texas); Maynard J A et al., Methods Mol. Biol. 2002 182: 149-63. This type of degeneration constitutes the minimal codon allowing introduction of the 19 amino acids. Differences in representation from one amino acid to another are attenuated (maximum ratio of 3 instead of 6 in the case of NNN codons). On the other hand, the frequency of stop codons is slightly higher (2 out of 32 codons instead of 3 out of 64). The effect on the quality of the codons in terms of expression cannot be generalized and depends on the host organism used for expression.

The use of NNN or NNK oligonucleotides therefore remains far from perfect. The ideal solution would be to have 19 oligonucleotides corresponding to the 19 possible substitutions at a given position. This approach would introduce the 19 possible substitutions at the same frequency, without introducing stop codons, and with perfect respect of the constraints of codon representation in the host organism.

If the 19 oligonucleotides are synthesized separately, there is a proportionate rise in the cost, since 19 separate oligonucleotides cost 19 times more than the degenerate oligonucleotide allowing to introduce all the mutations at once. In most cases, the benefit conferred by resolving the three shortcomings cited above does not justify the added cost.

An alternative solution makes it possible to introduce, at the mutant codon of each oligonucleotide, only the 20 desired codons, i.e., each of the 20 codons preferentially used by the organism in which the mutants are to be expressed. Said oligonucleotides can be synthesized by two methods: the first is based on fractionation on resin columns during oligonucleotide synthesis (U.S. 20030175887). This method is tedious and is not adapted to synthesis of large numbers of oligonucleotides. In a second approach, the 20 nucleotide triplets are individually synthesized by chemical methods in the form of phosphoramidites. The 20 trinucleotides are then combined in a mixture that can be used on an oligonucleotide synthesizer (just like any other nucleotide-phosphoramidite). Patent U.S. Pat. No. 5,869,644 describes the synthesis of such oligonucleotides for molecular biology. Patent U.S. Pat. No. 6,436,675 describes the use of such oligonucleotides in a context of recombination by gene synthesis. Nonetheless, trinucleotides-phosphoramidites are very complicated to synthesize and their-cost is excessive, ranging from 3 to 10 times higher than the cost of a simple or NNN degenerate oligonucleotide. This added cost is less than that of separately synthesizing 19 oligonucleotides, but is still open to criticism in view of the resultant benefit.

It may also be desirable to introduce in a residue not all possible substitutions, but only some of them. For example, one might want to conserve the chemical class of amino acid and substitute it only with an amino acid from the same class. One might also, for example, wish to avoid replacing hydrophobic residues by hydrophilic ones. In such case, semi-degenerate oligonucleotides can be used, that is to say, composed in reality of mixtures of 2 to 63 oligonucleotides differing only at the mutant codon, and allowing introduction of a diversity comprising from 2 to 18 different amino acids. The mutant codon in this case is composed of the combination of totally degenerate, single, and/or semi-degenerate bases, i.e., composed of a mixture of two or three bases. Oligonucleotide companies all offer the option of incorporating semi-degenerate bases. This “customized” diversity can turn out to be more difficult to introduce than total diversity since in some cases, it is not possible to design a single partially degenerate oligonucleotide to introduce the desired diversity, and two or three oligonucleotides need to be synthesized and used in a complementary fashion. Of course, it is conceivable that said semi-degenerate mutations can also be introduced by the nucleotide triplet approach described earlier. However, in this case it is necessary to prepare as many trinucleotide mixtures as there are different diversities to be introduced, and a minimum volume of each mixture must be prepared so as to have a vial that is full enough to be used on an oligonucleotide synthesizer. If one wants to introduce the same diversity at all target sites of the gene, this approach can be used. But if one wants to generate custom diversities, which differ from one reside to another, the high cost of trinucleotides and the need for a minimum volume of mixture are major obstacles.

The fifth approach for generating diversity is Massive Mutagenesis (Delcourt and Blésa, WO/0216606). This method allows directed mutations to be introduced not singly, but in a multiple and combinatorial manner. Said multiple mutations have specific characteristics as compared with single mutations: synergies between mutations might give a double mutant improved activity relative to the wild type molecule, whereas each of the two single mutations alone confers no improvement.

Massive Mutagenesis is a method based on the simultaneous use of a large number of oligonucleotides (more than 5 and preferably comprised between 50 and 5000), all with the same orientation, to prime the replication of a circular plasmid using a thermostable polymerase. Optionally, a thermostable ligase can also be added to the reaction to increase the mutation rate by actively ligating newly synthesized strands on the 5′ ends of the hybridized oligonucleotides. This method yields over 50% of molecules having incorporated at least one mutation. The mean number of mutations per mutant molecule can be modified at leisure, either by adjusting the concentrations of the various reagents or by performing the procedure several times in succession.

The Massive Mutagenesis reaction yields a mutant library, the diversity of which can in some cases comprise more than 108 different molecules.

An alternative approach to massive mutagenesis has been described for generating combinatorial diversity. It is based on complete synthesis of genes using oligonucleotides containing degenerate bases (Maxygen patent U.S. Pat. No. 6,579,678; Crea U.S. Pat. No. 5,798,208). However, this gene synthesis approach comes up against the problem of fidelity of oligonucleotide synthesis (approximately 0.5% misincorporation at each position), which is far below the fidelity of DNA replication by a polymerase (less than 0.01% misincorporation per position during PCR amplification). Thus, most of the synthesized genes contain, in addition to the target mutations, one or more secondary mutations, usually deletions of one or several bases. In most cases, these deletions shift the reading frame and make translation of the protein impossible. Said method of generating combinatorial diversity by complete synthesis therefore results in a very large proportion of useless mutants, thereby making it necessary to do more intensive screening to identify a positive mutant. In the case where the screening system is extremely efficient, as in mass selection approaches, the quality of diversity is of little importance. However, when screening requires considerable effort, it is preferable to use a technology that gives a higher rate of useful mutants. This is the case with Massive Mutagenesis, in which unwanted mutations are incorporated only very rarely.

In one of its applications, Massive Mutagenesis yields the entire set of alanine mutants (or any other given amino acid of a gene), that can be used to identify positions essential to protein activity. In this application, a library is obtained containing mutants which either have not integrated any mutation, or which have integrated one or more alanine substitutions of a codon. The activity of such mutants is measured individually, and the protein can be functionally mapped.

In a second application, Massive Mutagenesis generates a very large number of single or multiple mutants by introducing a variable diversity at certain sites of a gene. The number of target sites, the nature of the diversity, and the mean number of mutations per molecule can be adjusted at will. If a wide diversity is desired, oligonucleotides containing degenerate bases, such as described earlier, can be used. It is also possible to introduce only those substitutions that were preselected by bioinformatics (by modelling or by analysis of homologous natural sequences) and associated with an increased likelihood of conferring an improvement.

Out of all the mutagenesis technologies, Massive Mutagenesis is the only one that can produce customized diversity, i.e., a large number of molecules containing combinations of defined mutations obtained in a single reaction and in a short time, without the need to know sequences other than that of the gene to be mutated. When applied to the molecular evolution of proteins, this technology allows rational elements to be integrated into the introduced diversity, thereby increasing the frequency of positive mutants and enlarging the sequence space explored all while lowering the costs of screening.

Nevertheless, Massive Mutagenesis has two limitations:

First, the technology is based on the use of a large number of oligonucleotides, the costs of which can limit the use of this technology, when this number is high.

Secondly, when one wants to introduce wide diversity at several points, by using oligonucleotides containing degenerate bases (of the type NNN or NNK for example), representation biases of the different amino acids, described earlier in the case of directed mutagenesis, are exacerbated here. For example, the bias introduced at one site by a degenerate NNN oligonucleotide is a factor of 6 between tryptophane (Trp) and serine (Ser). In the case of double mutants, there is a 36-fold bias between the Trp-Trp combination and the Ser-Ser combination. The bias related to adaptation of certain codons to be expressed in the host organism, also described earlier in the case of directed mutagenesis, is also encountered in Massive Mutagenesis in a more amplified form.

These cost and quality limitations detract from the efficiency of the technology. The quality limitation can be resolved in part by an approach based on the use of trinucleotide cassettes, but as described earlier under directed mutagenesis, this approach offers only a partial solution; the very high cost of chemical synthesis of trinucleotides and the complexity of the approach (precluding the modulation at leisure of the diversity introduced at each position) also apply in the case of Massive Mutagenesis.

A technology that could overcome these two limitations of cost and quality would make it easier to obtain improved mutants and would therefore be economically interesting, principally in the field of industrial enzymes and therapeutic proteins; it would also facilitate certain basic research projects, particularly in the field of protein functional mapping.

### SUMMARY OF THE INVENTION

The invention has as its object a method for producing, directly in the form of libraries, single or multiple directed mutant polynucleotides of better quality and/or at lower cost as compared with the methods of the prior art.

In the Massive Mutagenesis method, the oligonucleotides used to introduce mutations are employed in the form of a library, each being present in a very low amount. Said oligonucleotides are synthesized and put back into solution individually, after which they are combined for use in the mutagenesis reaction which typically consumes 0.1 to 10 picomoles of each oligonucleotide. Now, the scale of synthesis of these oligonucleotides, even selecting the smallest possible scale available on commercial synthesizers, is several dozen nanomoles. Therefore only a small portion of each oligonucleotide is used. This wastefulness should be compared with the high cost of individually synthesizing the oligonucleotides in the implementation of Massive Mutagenesis technology.

The present invention relates to a method of mutagenesis characterized in particular by the use of a large number of oligonucleotides synthesized on a solid support, more particularly on oligonucleotide chips. Indeed, oligonucleotide mixtures generated by using DNA chips would cost forty times less than the same mixtures synthesized by the conventional approach of individually synthesizing the oligonucleotides.

The invention is further characterized by the use of a physical and/or chemical method allowing said oligonucleotides, once they have been synthesized on said solid support, to be cleaved from the support and placed in solution. More specifically, said oligonucleotides are obtained directly from the chip in the form of a mixture. In one embodiment, a chemical compound, which is labile under certain physicochemical conditions, is deposited on the solid support prior to the synthesis of the oligonucleotides. At the end of the synthetic reaction, the oligonucleotides are put in solution (in the form of a mixture) by subjecting the chip to the conditions associated with said lability.

The invention concerns a method for producing a library of mutant genes comprising the following steps:
a. Synthesizing on a solid support an oligonucleotide library comprising oligonucleotides complementary to one or several regions of one or several target genes and each comprising, preferably in their center, one or more mutations relative to the sequence of the target gene or genes;
b. Placing the oligonucleotide library obtained in step a) in solution; and,
c. Generating a library of mutant genes by using the oligonucleotide library in solution obtained in step b) and one or more templates containing said target gene or genes.

Preferably, in step c), the mutant gene library is generated by the Massive Mutagenesis method (described in particular in WO/0216606). More particularly, the invention concerns the aforementioned method, in which step c) comprises the following steps:
i. Providing one or more templates containing said target gene or genes;
ii. Contacting said template or templates with the oligonuclotide library synthesized in step a) in conditions allowing annealing of the oligonucleotides in the library to said template or templates so as to produce a reaction mixture;
iii. Carrying out replication of said template or templates in the reaction mixture through the use of a DNA polymerase;
iv. Eliminating the starting template or templates from the product of step iii) and thereby selecting newly synthesized DNA strands; and, optionally,
v. Transforming an organism with the DNA mixture obtained in step iv).

Preferably, the template is a circular nucleic acid, more particularly a plasmid. Alternatively, the template may be a linear nucleic acid. In a preferred embodiment, the template contains elements allowing the expression of said target gene or genes.

Preferably, the oligonucleotides of said library synthesized on the solid support are coupled to said solid support by means of a cleavable spacer molecule and said oligonucleotides are placed in solution by subjecting the oligonucleotides coupled to the solid support to conditions associated with cleavage of the spacer molecule. The spacer molecule can be cleaved in basic medium, by reaction to light, or by enzymatic reaction. However, the invention is not confined to this embodiment and encompasses any means of synthesis of an oligonucleotide library on a solid support allowing said oligonucleotide library to be subsequently placed in solution. More particularly, the solid support is a DNA chip. In a particular embodiment, said spacer molecule is cleavable in basic medium. For example, the basic medium is an ammonia solution. In a preferred embodiment, said spacer molecule is the compound represented by the following formula (compound A):

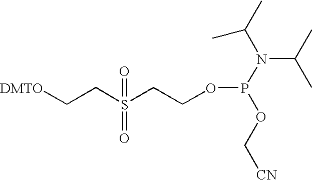

In a preferred embodiment, said spacer molecule is the compound represented by the following formula (compound B):

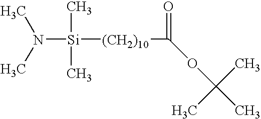

Preferably, each oligonucleotide in the library obtained in step b) is present in an amount comprised between 1 femtomole and 1 picomole.

In a particular embodiment, step iv) is carried out by means of a restriction enzyme specific for methylated DNA strands, preferably belonging to the group of enzymes: DpnI, NanII, NmuDI or NmuEI.

In a preferred embodiment, the oligonucleotides synthesized in step a) are all complementary to a same target gene.

Preferably, all the oligonucleotides complementary to a same target gene are complementary to the same strand of said target gene.

In a first preferred embodiment, the oligonucleotide library synthesized in step a) contains oligonucleotides bearing mutations allowing to introduce all possible substitutions at each codon of said target gene or genes. In a second preferred embodiment, the oligonucleotide library synthesized in step a) contains oligonucleotides bearing mutations allowing to introduce a same amino acid, preferably an alanine, at each codon of said target gene or genes.

Preferably, the synthesis of the oligonucleotide library on the solid support is carried out by any suitable method of oligonucleotide synthesis on chips well-known by the man skilled in the art, among which are the above-described methods.

Preferably, said organism in step v) is a bacterium or a yeast.

In a first embodiment, the DNA polymerase is a thermosensitive polymerase. For example, it may be selected in the group consisting of E. coli T4 DNA polymerase or else the Klenow fragment of E. coli polymerase. In a second embodiment, the DNA polymerase is a thermostable polymerase. For example, it may be selected in the group consisting of Taq, Pfu, Vent, Pfx or KOD polymerases.

In addition, the invention relates to a method of directed mutagenesis comprising the steps of the method for producing a library of mutant genes according to the invention.

The invention also relates to a method of mutagenesis of a target protein or of several target proteins, characterized in that it comprises preparing a mutant gene expression library from a target gene coding for said protein, or from several target genes coding for said proteins, by the method of producing a mutant gene library according to the invention, then expressing said mutant genes to produce a mutant protein library.

The invention relates to a method of evolution of a gene or a protein comprising preparing a library of mutant genes or mutant proteins according to the invention then selecting the mutant genes or mutant proteins having the desired property.

The invention relates to a solid support carrying an oligonucleotide library comprising oligonucleotides complementary to one or several regions of one or several target genes and each comprising, preferably in their center, one or more mutations relative to the sequence of the target gene or genes. In a first preferred embodiment, the oligonucleotide library contains oligonucleotides bearing mutations allowing to introduce all possible substitutions at each codon of said target gene or genes. In a second preferred embodiment, the oligonucleotide library contains oligonucleotides bearing mutations allowing to introduce a same amino acid, preferably an alanine, at each codon of said target gene or genes. Preferably, the oligonucleotides of said library are coupled to said solid support by means of a cleavable spacer molecule. For example, the spacer molecule can be cleavable in basic medium, by reaction to light, or by an enymatic reaction. In a particular embodiment, said spacer molecule can be cleaved in basic medium. For example, the basic medium is an ammonia solution. In a preferred embodiment, said spacer molecule is compound A. In this embodiment, said spacer molecule is preferably compound B.

### DETAILED DESCRIPTION OF THE INVENTION

DNA chips are composed of a solid support measuring a few square millimeters or centimeters on which a large number of different DNAs are deposited in an orderly arrangement (Heller M J et al., Ann. Rev. Biomed. Eng. 2002; 4: 129-53). The first functional DNA chips were homemade in molecular biology laboratories. In these first experiments, the DNA applied on the chips was produced by biochemical synthesis, for example PCR fragments of the yeast genome ORFs (Schena M et al., Science. 1995.270 (5235): 467-70; Spellman P T et al., Mol. Biol. Cell. 1998. 9(12): 3273-97).

Today, in the most common case, these DNAs are chemically synthesized oligonucleotides from 5 to 200 bases long, typically from 15 to 100 bases. Hybridization of nucleic acids from various sources (cDNA from different tissues, genomic DNA, etc.) on these oligonucleotide chips (hereinafter called “DNA chips” or simply “chips”) provides information, particularly in the field of transcriptome analysis and detection of polymorphisms (for a set of complete reviews see Nature Genetics volume 32 supplement pp. 461-552). These methods are now routinely used in a great number of research and medical diagnostics laboratories the world over for massive, semiquantitative and parallel evaluation of the nucleic acid concentrations in nucleic acid mixtures.

Two major types of technology enable production of said chips. In a first approach, the different oligonucleotides are synthesized chemically by using phosphoramidites and a conventional oligonucleotide synthesizer. Said oligonucleotides are then deposited on a slide, for example, by spotting or by microfluidic technologies similar to those used in ink-jet printers. A second approach is to manufacture the chips by synthesizing the oligonucleotides directly on the slide. Parallel in situ synthesis of a large number of oligonucleotides is made possible by special nucleotide coupling chemistry which depends on the presence of light or by classical chemistry by a well-localized addressing of the nucleotides (e.g., piezo, microvalves, or any system of spraying) into defined areas (WO 95/35505, WO 02/26373). In the first embodiment, selective light exposure of some of the “pixels” on the chip, in the presence of one of the four bases, induces a photoactivated reaction through which said base is coupled to only some of the oligonucleotides being synthesized. In the next step, selective light exposure of other pixels, in the presence of another base, allows elongation of another subset of these oligonucleotides.

In the manufacture of oligonucleotide chips, 10² to 10⁶ (typically: 10³ to 10⁵) oligonucleotides of different sequence are therefore synthesized in parallel, at a very small scale of synthesis (less than one picomole in most cases). There are several techniques by which to accurately create a selective lighting. A first method makes use of photolithographic masks (Pease, A C et al. Proc. Natl. Acad. Sci. USA, 91, 5022-5026 and patents held by Affymetrix Inc.) which are costly but useful when one wants to produce a large series of identical chips and which have excellent contrast ratio. A second method uses digital micromirror devices (DMD; Sangeet Singh-Gasson et al., Nat. Biotech. 1999 17 (10): 974-978; LeProust E. et al., J. Comb. Chem., 2, 349-354 and WO9942813; WO0047548; U.S. Pat. No. 6,271,957). Although the contrast ratio is lower, this type of technique has the advantage of very high flexibility, making it particularly useful for small-scale manufacture of custom chips at a reasonable price. Other techniques bypassing the use of permanent masks, and using for instance liquid crystal displays, have been described (U.S. Pat. No. 5,424,186). Another methods are also described in WO 95/35505 and WO 02/26373.

This miniaturized and parallel approach has made it possible to radically cut the costs of oligonucleotide synthesis, provided that the latter can be used in the form of a mixture in which each oligonucleotide is present in only a small amount. By conventional chemical synthesis, the cost of oligonucleotide synthesis is, to a first approximation, proportional to the number of oligonucleotides and increases with their length. By the chip-based approach, and with the aforementioned reservations, the cost of synthesizing an oligonucleotide mixture depends solely on their length and becomes flat rate (per chip). Today, it costs roughly 2000 euros to synthesize a chip containing 8000 different oligonucleotides of about thirty bases each. By way of comparison, it would cost about 80,000 euros to synthesize these 8000 oligonucleotides individually by the conventional approach, i.e., on a synthesizer.

Thus, oligonucleotide mixtures generated by using DNA chips would cost forty times less than the same mixtures synthesized by the conventional approach of individually synthesizing the oligonucleotides.

More specifically, the inventive method is characterized by the following sequence of steps:

a) A mutagenesis strategy for one or more target genes is designed. The final objective of said strategy may be either to improve some of the properties of said target gene, or to obtain scientific data on this gene, in particular so as to characterize the amino acids directly related to its function. One or more mutations can be designed for a target codon.

b) Based on this strategy, a set of mutant oligonucleotides is designed. Each oligonucleotide contains one or more mutations, preferably located in its center. The number of mutant oligonucleotides is generally equal to the sum of the different mutations to be introduced at each codon. Advantageously, the oligonucleotides are all homologous to the same strand of the template.

c) The mutant oligonucleotides designed in step b) are synthesized by using a chip-based approach of oligonucleotide synthesis. Preferably, the approach based on the use of micromirrors is used, since it is better suited to custom synthesis of large numbers of oligonucleotides. Preferably, prior to synthesizing the oligonucleotides, a chemical compound serving as a spacer, which is labile under certain physicochemical conditions, will have been deposited on the chip.

d) The oligonucleotides are released from their support, to be placed in solution. Preferably, the oligonucleotides are released by applying the physicochemical conditions associated with lability of the chemical spacer. Each oligonucleotide has to be present in an amount comprised between 1 femtomole and 1 picomole.

e) Separately, a sufficient amount of one or more templates (plasmids or linear templates, preferably plasmids) is prepared, containing the target gene or genes and optionally one, two or more selectable markers (for example, antibiotic resistance genes). Preferably, the template, preferably the plasmid, also contains an antibiotic resistance gene and the promoter driving expression of said resistance gene, an origin of replication, and optionally a promoter driving expression of the target gene, as well as all the maturation sequences (poly-A, splicing signals, etc.) allowing to optimize the expression of a mature protein, from the target gene, in the chosen organism.

f) A reaction mixture containing the template prepared in step e) and the oligonucleotide mixture obtained in step d) is prepared.

g) The reaction mixture is subjected to an elevated temperature (greater than 80° C. and preferably approximately 94° C.) so that single-stranded DNA will temporarily be present.

h) The temperature is lowered to a value comprised between 0 and 60° C., and preferably between 20 and 50° C., so that each oligonucleotide present in the mixture anneals to its site of homology in the target gene or in one of the target genes.

i) The reaction mixture is subjected to a temperature compatible with the activity of a DNA polymerase, which is added to the reaction mixture with a sufficient amount of each nucleotide triphosphate, buffers and required cofactors. The reaction is carried out for a sufficient time to ensure complete replication of the template.

j) Any suitable method is used to eliminate the starting templates and thereby select the newly synthesized DNA strands generated in step l). Advantageously, this selection step is carried out by means of a restriction enzyme specific for methylated DNA strands, and preferably belonging to the group of enzymes: DpnI, NanII, NmuDI and NmuEI. Optionally, the DNA fragment synthesized in step j) is used as an insert to be cloned into a previously linearized plasmid, for example using the so-called “TA-cloning” approach.

k) The reaction mixture obtained in step j) is transformed into a suitable organism such as transformation-competent yeast or bacteria, for example by electroporation or heat shock.

Avantageously, the oligonucleotides are designed, in step b), so that all the oligonucleotides homologous to a same target gene are homologous to the same strand of said target gene.

Preferably, the oligonucleotide library is synthesized on a same solid support. In another alternative, the oligonucleotides in the library having an A in 3′ position are synthesized on a same solid support, those having a C in 3′ position are synthesized on another solid support, those having a G in 3′ position are synthesized on yet another solid support, and finally those having a T in 3′-position are synthesized on another solid support. Oligonucleotide library is understood to mean a composition comprising at least 2, 10, 20 or 50 different oligonucleotides. Said oligonucleotide library preferably comprises more than 50, 100, 200, 500, 1000, or 5000 different oligonucleotides. Preferably, the solid support is a chip. In one embodiment, the solid support is glass. However, other types of supports are also encompassed in the invention.

In a preferred embodiment, a chemical compound playing the role of spacer between the solid support or slide and the oligonucleotides (a “spacer”) is deposited on the solid support or slide prior to synthesis of the oligonucleotides as described in step c). Said spacer also has the characteristic of being labile under certain physicochemical conditions. For example, the chemical compound can be compound A represented by the formula:

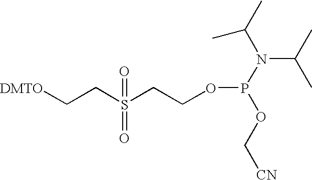

The linkage between the compound and the synthesized oligonucleotide is cleavable in basic conditions.

In another example, the chemical compound can be compound B represented by the formula:

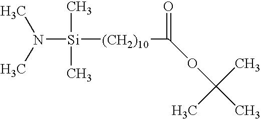

Said compound can be cleaved by ammonia.

In a preferred embodiment, the oligonucleotides are placed in solution in step d) by applying the conditions of lability of the chemical spacer, for example in basic conditions for compound A or compound B. When compound A is used, the oligonucleotides obtained are phosphorylated in 3′. The method optionally comprises a “deprotection” step, i.e., eliminating said phosphate group present at the 3′ end.

The amount of template (preferably a plasmid) from step e) is preferably comprised between 10 ng and 100 μg, more preferably comprised between 100 ng and 10 μg and even more preferably comprised between 100 ng and 1 μg.

Preferably, the template is a plasmid.

In a first embodiment, the reaction mixture of step i) contains a thermosensitive polymerase. For example, and not by way of limitation, E. coli T4 polymerase is used, or else only the Klenow fragment of E. coli polymerase.

In a second embodiment, the reaction mixture of step i) contains a thermostable polymerase with or without specific reading fidelity. For example, and not by way of limitation, the Taq, Pfu, Vent, Pfx or KOD polymerase is used. It is also possible to use a mixture of two or more of such enzymes (for example 1 unit of Pfu polymerase and 5 units of Taq polymerase).

In a particular embodiment, steps g), h) and i) are carried out several times so as to constitute several temperature cycles. In such case, the polymerase used is preferably thermostable, so that it is not necessary to add polymerase at each cycle.

In a particular embodiment, a ligase as well as buffers and required cofactors are added to the reaction mixture of step i). In such case, the oligonucleotides of the mixture such as described in d) incorporate a phosphoric acid group in 5′. Said phosphoric acid group can have been incorporated directly during oligonucleotide synthesis. Preferably, the oligonucleotides are synthesized normally and then 5′ phosphorylated with the help of a kinase (for example, T4 polynucleotide kinase), after being synthesized.

In the case where several temperature cycles g), h), i) are carried out, and where the polymerase used is thermostable, it is preferably to use a ligase which is also thermostable, so that it is not necessary to add this enzyme at each cycle. For example, and not by way of limitation, Taq Ligase, Tth ligase or Amp ligase is used.

In the case where a single temperature cycle is carried out and where the polymerase used is thermosensitive, it is preferable to use a ligase which is also thermosensitive, or at least partially active at the same temperature as the polymerase used.

The invention can additionally comprise the following step:

l) The bacteria are plated on a medium containing a selection agent so as to select those bacteria having integrated a template, preferably a plasmid, potentially containing a mutant target gene.

The invention can additionally comprise the following step:

m) The bacterial colonies obtained in l) are isolated and inoculated into a selective nutrient medium.

The invention can additionally comprise the following step:

n) From the different cultures prepared in m), the same number of DNA preparations, preferably plasmidic, are prepared, each corresponding to an isolated clone containing a target gene potentially mutated at one or more positions.

The invention can additionally comprise the following step:

o) The DNA preparation, preferably plasmidic, obtained in n) is used to express the corresponding protein. To do this, the plasmid DNA is introduced into a prokaryotic or eukaryotic organism adapted to expression. For example, and not by way of limitation, bacteria, yeast, fungus, insect cell, plant cells, mammalian cells are used. Expression may be constitutive or inducible (for example, by temperature, a biochemical inducer). In the case of inducible expression, conditions are used which enable induction and expression. Alternatively, the corresponding protein can be produced by using an existing in vitro transcription/translation system (Betton J M., Curr. Protein Pept. Sci. 2003. 4(1): 73-80). In the case where translation takes place in vitro, an in vitro step of protein maturation or folding can be added after synthesis of the protein (GAO Y G et al., Biotechnol. Prog. 2003. 19(3): 915-20; Kosinski-Collins M S et al., Protein Sci. 2003.12(3): 480-90). In the case where translation takes place in a cell, as in the case where translation takes place in vitro, it is possible, if one uses a non-standard genetic code when designing the oligonucleotides used to introduce the mutations, to integrate non-natural amino acids (Chin J W et al., Science 2003. 301(5635): 964-7; Hohsaka T et al., Nucleic Acids Res. Suppl. 2003(3): 271-2; Taki M et al., Nucleic Acids Res. Suppl. 2001; (1): 197-8; I Hirao et al., Nat. Biotech. 20, 177-182).

The invention can additionally comprise the following step:

p) The activity (or other parameters such as stability, thermostability, substrate specificity, activity in the presence of an inhibitor, etc.) of the protein obtained by lysis or without lysis of the cultures obtained in m) or n) is measured directly or indirectly, and said activity is compared with that of the protein produced under the same conditions from DNA, preferably plasmidic, containing the non-mutant target gene. When said measurements reveal a difference considered to be significant, the mutant molecules can eventually be sequenced so as to identify the position of the mutation underlying said modification of activity.

In a particular embodiment, the library produced by the method is subjected to a so-called selection technique, where the gene products (phenotypes), which have previously been related to the nucleic acids encoding them (genotypes), are all sorted at the same time (in bulk). In this case the first steps a) to k) of the method remain unchanged but steps l), m), n), o) and p) described hereinabove are deleted and replaced by the following steps:

l′) the cells from step k) are cultured in a suitable liquid selection medium.

m′) an existing method of selection is used. For example, and not by way of limitation, the survival of transformed cells on some minimum medium can be used, one can also used a “phage display”, “cell-surface display”, “ribosome display”, mRNA-peptide fusion, selection in emulsion or protein fragment complementation test.

n′) if necessary, the selected nucleic acids are recloned in the initial plasmid then the plasmids are reused in step f) for a new round of the method. Alternatively, said nucleic acids are subjected to secondary screening and/or sequencing.

In a particular embodiment, one uses in step f) not the template prepared in e) but the DNA, preferably plasmidic, prepared from cells transformed in step k) during a previous round of the method. In this way it is possible to carry out several (typically: 2 to 20) successive rounds of mutagenesis; at each round, the percentage of mutant genes in the library and the mean number of mutations per molecule increase.

In a particular embodiment, one uses in step f) not the template prepared in e) but the DNA, preferably plasmidic, prepared from cells which expressed an improved protein activity in step p) during a previous round of the method. The mutations to be introduced into these already mutated and already improved molecules can be identical or not to the mutations introduced in the first round of the method. In this way it is possible to carry out several rounds of molecular evolution by mutation-selection (or screening).

In a particular embodiment, the method is characterized by evolution not of the proteins but of one or more nucleic acids (DNA or RNA).

In a particular embodiment, the oligonucleotides are designed so as to introduce not point substitutions but deletions of several bases (1 to 20, typically 1 to 9) or insertions of several bases (typically 1 to 9).

### Embodiment #1

### High Temperature

In a first embodiment, a combinatorial mutant library is produced from a gene and the inventive method is characterized by the following sequence of steps:

a) A mutagenesis strategy is designed for a target gene composed of n codons, with n preferably comprised between 50 and 5000. This strategy can concern either all the n codons of the target gene, or only a portion of these n codons.

b) Based on this strategy, a set of mutant oligonucleotides is designed, preferably having a size comprised between 15 and 45 nucleotides and each being homologous to a region of the target gene.

c) The corresponding mutant oligonucleotides designed in b) are synthesized by using a chip-based method of oligonucleotide synthesis.

d) The oligonucleotides are released from their support, so as to obtain a mixture of oligonucleotides in solution.

e) Separately, a template, preferably a plasmid, is prepared, containing the target gene, using a suitable preparation system (mini-, midi- or maxi-prep systems available from specialized companies (Qiagen, Macherey-Nagel, etc. . . . ).

f) A reaction mixture is prepared containing the template prepared in e) and the oligonucleotide mixture obtained in d), at a concentration such that the ratio between the number of template molecules and the number of molecules of each mutant oligonucleotide is comprised between 0.01 and 100, preferably between 0.1 and 10. A thermostable polymerase is added, together with all the necessary reagents for replication of the template from the mutant oligonucleotides: reaction buffer, nucleotide triphosphates in sufficient amount, any required cofactors.

g) The mixture is subjected to an elevated temperature (greater than 80° C. and preferably approximately 94° C.), for at least one second and preferably for approximately one minute, so that single-stranded DNA will temporarily be present.

h) The temperature is lowered to a value comprised between 0 and 60° C. and preferably comprised between 20 and 50° C., for at least one second and preferably for approximately one minute, so that the oligonucleotides present in the mixture anneal to their site of homology in their target gene.

i) The reaction mixture is subjected to a temperature of approximately 68 to 72° C., which allows optimal activity of the polymerase, for a sufficient time to allow complete replication of the template, preferably the plasmid, and calculated according to the rate of synthesis of the polymerase (in bases per minute) and the size of the template, preferably the plasmid, containing the target gene.

Steps g), h), and i) are repeated, preferably by using a thermocycler, so that the temperature cycles can be performed automatically.

j) Any suitable method is used to select newly synthesized DNA strands generated during step g) from the starting templates.

k) The reaction mixture obtained in j) is transformed into a suitable organism, such as yeasts or bacteria, rendered transformation-competent.

In step f), a thermostable ligase like Taq Ligase or Tth ligase can optionally be added. Advantageously in such case, the oligonucleotides will have been 5′ phosphorylated by means of a kinase prior to their use.

This embodiment of the invention can additionally contain one or more of the steps l), m), n), o), or p) described hereinabove.

### Embodiment #2

### Low Temperature

In a second embodiment, the inventive method is characterized by the following sequence of steps:

A strategy is determined, the oligonucleotides are designed, synthesized on a solid support, released and, independently, a sufficient amount of template containing the target gene is prepared, such as described in steps a), b), c), d), and e) and the previous example. The subsequent steps are:

f) A reaction mixture is prepared containing the template prepared in e) and the oligonucleotide mixture obtained in d), at a concentration such that the ratio between the number of template molecules and the number of molecules of each mutant oligonucleotide is comprised between 0.01 and 100, preferably between 0.1 and 10.

g) The mixture is subjected to an elevated temperature (greater than 80° C. and preferably approximately 94° C.), for at least one second and preferably for approximately one minute, so that single-stranded DNA will temporarily be present.

h) The temperature is lowered to a value comprised between 0 and 60° C. and preferably comprised between 20 and 50° C., for at least one second and preferably for approximately one minute, so that the oligonucleotides present in the mixture anneal to their site of homology in their target gene.

i) A thermosensitive polymerase is added, for example T4 polymerase, together with all the necessary reagents for replication of the template from the mutant oligonucleotides: reaction buffer, nucleotide triphosphates in sufficient amount, any required cofactors. The reaction mixture is subjected to a temperature of approximately 37° C., which allows optimal activity of the T4 polymerase, for a sufficient time to allow complete replication of the plasmid, and calculated according to the rate of synthesis of the polymerase (in bases per minute) and the size of the plasmid containing the target gene.

Steps g), h) and i) can possibly be repeated one or more times.

j) Any suitable method is used to select newly synthesized DNA strands generated during step g) from the starting templates.

k) The reaction mixture obtained in j) is transformed into a suitable organism, such as yeasts or bacteria, rendered transformation-competent.

In step f), a ligase can be added, preferably thermosensitive and in any case active at the activity temperature of the polymerase used, such as T4 ligase. Advantageously in such case, the oligonucleotides will have been 5′ phosphorylated by means of a kinase prior to their use.

In a particular embodiment, in step f) one uses not the template prepared in e) but the DNA, preferably plasmidic, prepared from cells transformed in step k). In this way it is possible to carry out two or more successive rounds of mutagenesis: at each round, the percentage of mutant genes in the library and the mean number of mutations per molecule increase.

This embodiment of the invention can additionally contain one or more of the steps l), m), n), o), or p) described hereinabove.

### Embodiment #3

### False Multigene

In a particular embodiment, the oligonucleotides corresponding to several genes are synthesized simultaneously, then said oligonucleotides are separated (for example, by chromatography or by capillary electrophoresis, on the basis of their mass, if oligonucleotides of different length are designed for each gene, for example oligonucleotides of length 18 for gene 1, 20 for gene 2, 22 for gene 3, . . . , 36 for gene 10). These different oligonucleotide mixtures can then be used normally in one of the embodiments described hereinabove.

### Embodiment #4

### Pooled Multigene

In a fourth embodiment, several genes are mutated simultaneously in a single reaction mixture containing all the oligonucleotides allowing the desired mutations to be introduced in all the genes. The inventive method is characterized by the following sequence of steps:

a) Of interest is a set of target genes Gᵢ, with i ranging from 1 to g, and g preferably comprised between 2 and 1000. Each of said target genes Gᵢ is composed of nᵢ codons, with nᵢ preferably comprised between 50 and 5000. For each gene Gᵢ, a mutagenesis strategy is designed. The strategy corresponding to each gene Gᵢ can concern either all nᵢ codons, or only a portion of said codons.

b) Based on each strategy, a set of mutant oligonucleotides is designed for each corresonding gene Gᵢ, preferably having a size comprised between 15 and 45 nucleotides and each being homologous to a region of the gene Gᵢ. It is possible that the sequences of two or more genes have a high degree of similarity in certain regions and therefore that some of the oligonucleotides designed to introduce mutations in one of said genes hybridize not only to the desired gene but also to one or more other genes, thereby creating unwanted mutations. This embodiment therefore assumes that mixtures of genes with a very high degree of sequence homology will be avoided and, in any case, that potential cross-hybridization phenomena will be taken into account in the design of the oligonucleotides. To aid in the design of oligonucleotides in this embodiment of the method, it is possible to use existing algorithms or software to optimize the oligonucleotide sequences and avoid such cross-hybridization phenomena. These programs, currently dedicated to the design of oligonucleotide chips for transcriptome analysis or multiplex PCR, can be used as is, with minor adaptations (see for example Emrich S J, Nucleic Acids Res. 2003. 31(13): 3746-50; Xu D., Bioinformatics 2002 18(11): 1432-7).

c) The corresponding mutant oligonucleotides designed in b) are synthesized on a chip.

d) The oligonucleotides are released from their support, so as to obtain a mixture of oligonucleotides in solution.

e) Independently, each of g templates, preferably plasmids, is prepared separately, each containing one of the target genes Gᵢ. The amount of each template, preferably of each plasmid, prepared is preferably comprised between 10 ng and 10 μg. In a particular embodiment, each template contains several selectable markers, so as to be able to grow clones containing said template, in a suitable selection media, to the exclusion of all other clones. (For example, it is possible to recover any one of four given templates if the following markers are introduced into their sequence: chloramphenicol and ampicillin for the first, chloramphenicol, ampicilin and tetracycline for the second, chloramphenicol and tetracycline for the third, chloramphenicol alone for the fourth).

f) A reaction mixture is prepared containing all the templates prepared in e) and the oligonucleotide mixture obtained in d), at concentrations such that the ratio between the number of template molecules and the number of molecules of each corresponding mutant oligonucleotide is comprised, for each template, between 0.01 and 100, preferably between 0.1 and 10. A thermostable polymerase is added, together with all the necessary reagents for replication of the template from the mutant oligonucleotides: reaction buffer, nucleotide triphosphates in sufficient amount, any required cofactors.

g) The mixture is subjected to an elevated temperature (greater than 80° C. and preferably approximately 94° C.), for at least one second and preferably for approximately one minute, so that single-stranded DNA will temporarily be present.

h) The temperature is lowered to a value comprised between 0 and 60° C. and preferably comprised between 20 and 50° C., for at least one second and preferably for approximately one minute, so that the oligonucleotides present in the mixture anneal to their site of homology in their target gene.

i) The reaction mixture is subjected to a temperature of approximately 68 to 72° C., which allows optimal activity of the polymerase, for a sufficient time to allow complete replication of the template, preferably the plasmid, and calculated according to the rate of synthesis of the polymerase (in bases per minute) and the size of the plasmid containing the target gene.

Steps g), h), and i) are repeated, preferably by using a thermocycler, so that the temperature cycles can be performed automatically.

j) Any suitable method is used to select newly synthesized DNA strands generated during step g) from the starting templates.

k) The reaction mixture obtained in j) is transformed into a suitable organism, such as yeasts or bacteria, rendered transformation-competent.

In step f), a thermostable ligase like Taq Ligase or Tth ligase can optionally be added. Advantageously in such case, the oligonucleotides will have been 5′ phosphorylated by means of a kinase prior to their use.

This embodiment of the invention can additionally contain one or more separate steps for the different genes Gᵢ.

One possibility is to plate the cells from step k) on a culture dish containing a selective medium, then to subculture these clones or a portion thereof in liquid medium followed by a PCR reaction on each culture using a set of oligonucleotides designed so that the size of the resulting product indicates the gene carried by the plasmid of the corresponding clone.

Alternatively, the cells from step k) are plated on a culture dish containing a selective medium, subcultured in liquid medium and each of the cultures is subjected to a set of g PCR reactions each using two oligonucleotides designed so that, for each clone, the existence of a product in one of g PCR reactions, and of no product in the other (g-1) reactions, indicates the gene carried by the plasmid of the corresponding clone.

Alternatively, the cells from step k) are cultured all together in a selective liquid medium, g PCR reactions of the preparative PCR type are then carried out on these cultures so as to amplify at each round a portion of the sequence of the plasmids corresponding to a single one of the g genes. The g PCR products are then purified separately (for example with a kit using a column or after loading on a gel with a suitable kit) to yield in linear form g libraries each corresponding to one of the g genes which were mutated. These linear libraries are cloned separately by conventional methods into the starting plasmids or into other suitable plasmids, then transformed, plated on solid medium so as to isolate clones and then screened. Alternatively, these linear libraries can be cloned separately then transformed, expressed and subjected to a selection.

Alternatively, in the case where the plasmids used in step e) each contain a set of selectable markers in a unique combination, the cells from step k) are plated on g different culture dishes each containing a combination of selection agents allowing the growth of only those cells containing a particular combination of selectable markers and therefore yielding in each dish clones containing just one of the Gᵢ genes.

Alternatively the cells from step k) are plated on a culture dish containing a selective medium, each of the independent clones obtained is subcultured in liquid medium, the plasmid DNA is prepared from each of these cultures and sequenced. From the sequencing results, one can determine for each clone which gene among the g genes is present and one has information on all or some of the mutations introduced into the sequence of said gene. The cultures performed before sequencing of each clone are then used for a screening test, and therefore a set of data is available of the type (mutant gene, sequence, result of screening test). The cultures performed before sequencing can also be mixed according to the gene they contain as indicated by the sequencing so as to recover libraries corresponding to each gene, which can then be screened or selected.

Alternatively, the plasmid DNA from all the cells from step k) is isolated then subjected in parallel to g multiple enzymatic digestions Rᵢ (i=1, 2 . . . g) by restriction enzymes. Each reaction Rᵢ (i=1, 2 . . . g) is designed so as to linearize each time all the plasmids except the plasmids containing the gene Gᵢ. After each reaction Rᵢ (i=1, 2 . . . g), the plasmids are used to transform bacterial or yeast cells and only circular plasmids, therefore only plasmids containing versions of the gene Gᵢ, are efficiently transformed. This approach may or may not be possible depending on the type of plasmid and gene used. This approach follows directly from differential multiple digestion (WO9928451).

In a particular embodiment, in step f) one uses not the template prepared in e) but the DNA, preferably plasmidic, prepared from cells transformed in step k). In this way it is possible to carry out two or more successive rounds of mutagenesis: at each round, the percentage of mutant genes in the library and the mean number of mutations per molecule increase.

### Embodiment #5

### Parallel Multigenes

In a fifth embodiment, several genes are mutated independently in parallel, but the oligonucleotides allowing the introduction of mutations in a set of g genes (g being typically comprised between 2 and 1000, preferably between 2 and 50) are synthesized simultaneously on the same chip.

In this embodiment, the inventive method is characterized by the following sequence of steps:

a) Of interest is a set of target genes Gᵢ (i=1, 2 . . . g) with g comprised between 2 and 1000. Each of said target genes Gᵢ is composed of nᵢ codons, with nᵢ preferably comprised between 50 and 5000. For each gene Gᵢ, a mutagenesis strategy is designed. The strategy corresponding to each gene Gᵢ can concern either all nᵢ codons, or only a portion of said codons.

b) Based on each strategy, a set of mutant oligonucleotides is designed for each corresponding gene Gᵢ, preferably having a size comprised between 15 and 45 nucleotides and each being homologous to a region of the gene Gᵢ. It is possible that the sequences of two or more genes have a high degree of similarity in certain regions and therefore that some of the oligonucleotides designed to introduce mutations in one of said genes hybridize not only to the desired gene but also to one or more other genes, thereby creating unwanted mutations. This embodiment therefore assumes that mixtures of genes with a very high degree of sequence homology will be avoided and, in any case, that potential cross-hybridization phenomena will be taken into account in the design of the oligonucleotides. Existing algorithms and software for optimizing oligonucleotide sequences and avoiding such cross-hybridization phenomena during design of oligonucleotide chips for transcriptome analysis or multiplex PCR can be used, with minor adaptations, to assist in the design of oligonucleotides in this embodiment of the method (par example: Emrich S J Nucleic Acids Res. 2003 Jul. 1; 31 (13): 3746-50; Xu D. Bioinformatics. 2002 November; 18(11): 1432-7). The oligonucleotides corresponding to each gene may or may not have different lengths between themselves and may or may not have lengths that differ from the oligonucleotides corresponding to the other genes.

c) The corresponding mutant oligonucleotides designed in b) are synthesized on a DNA chip.

d) The oligonucleotides are released from their support, so as to obtain a mixture of oligonucleotides in solution.

e) Independently, each of g templates, preferably plasmids, is prepared separately, each containing one of the target genes Gᵢ. Preferably, the template also contains an antibiotic resistance gene and the promoter driving expression of said resistance gene, an origin of replication, and optionally a promoter driving expression of the target gene as well as all the maturation sequences (polyA, splicing signals, etc.) allowing optimal expression of a mature protein, from the target gene, in the chosen organism.

f) g reaction mixtures are prepared. The reaction mixture Mᵢ contains the template, preferably the plasmid, carrying gene Gᵢ and the oligonucleotide mixture obtained in d), at a concentration such that the ratio between the number of template molecules and the number of molecules of each mutant oligonucleotide is comprised between 0.01 and 100, preferably between 0.1 and 10. A thermostable polymerase is added together with all the necessary reagents to carry out replication of the template from the mutant oligonucleoties: reaction buffer, nucleotide triphosphates in sufficient amount, any required cofactors.

g) Each mixture Mᵢ is independently subjected to an elevated temperature (greater than 80° C. and preferably approximately 94° C.) for at least one second and preferably for approximately one minute, so that single-stranded DNA will temporarily be present.

h) The temperature is lowered to a value comprised between 0 and 60° C. and preferably comprised between 20 and 50° C., for at least one second and preferably for approximately one minute, so that the oligonucleotides present in the mixture anneal to their site of homology in their target gene.

i) Each reaction mixture Mᵢ is subjected to a temperature of approximately 68 to 72° C., which allows optimal activity of the polymerase, for a sufficient time to allow complete replication of the template, preferably the plasmid, and calculated according to the rate of synthesis of the polymerase (in bases per minute) and the size of the plasmid containing the target gene.

Steps g), h), and i) are repeated, preferably by using a thermocycler, so that the temperature cycles can be performed automatically.

j) Any suitable method is used to select the newly synthesized DNA strands generated in step i) from the starting templates. Advantageously, this selection step is carried out by means of a restriction enzyme specific for methylated DNA strands, and preferably belonging to the group of enzymes consisting of DpnI, NanI, NmuDI and NmuEI.

k) The reaction mixtures obtained in j) are transformed into a suitable organism, such as yeasts or bacteria, rendered transformation-competent.

In step f), a thermostable ligase like Taq Ligase or Tth ligase can optionally be added. Advantageously in such case, the oligonucleotides will have been 5′ phosphorylated by means of a kinase prior to their use.

In a particular embodiment, in step f) one uses not the templates prepared in e) but the DNA, preferably plasmidic, prepared from cells transformed in step k). In this way it is possible to carry out two or more successive rounds of mutagenesis: at each round, for each gene the percentage of mutant genes in the corresponding library and the mean number of mutations per molecule increase.

### Embodiment #6

### Mutagenesis and Selection by Plasmid Display

In a sixth embodiment, a combinatorial mutant library is created from a gene and said library is selected by “plasmid display” (Speight R E et al., Chem. Biol. 2001 8(10): 951-65; Zhang Y et al., J. Biochem. (Tokyo) 2000 June; 127(6): 1057-63; Cull M G et al.,
15. Proc. Natl. Acad. Sci. USA. 1992 Mar. 1; 89(5): 1865-9).

In this embodiment, the inventive method is characterized by the following sequence of steps:

a) A mutagenesis strategy is designed for a target gene composed-of n codons, with n preferably comprised between 50 and 5000. Said strategy can concern either all n codons, or only a portion of said codons.

b) Based on this strategy, a set of mutant oligonucleotides is designed, preferably having a size between 15 and 45 nucleotides and each being homologous to a region of the target gene.

c) The corresponding mutant oligonucleotides designed in b) are synthesized by using any type of chip-based method of synthesis.

d) The oligonucleotides are released from their support, so as to obtain a mixture of oligonucleotides in solution.

e) Independently, a matrix, preferably a plasmid, containing the target gene is prepared. The matrix also contains, flanking the target gene (upstream or downstream) and under control of the same promoter (so as to produce a fusion protein), the gene encoding a protein P¹ which has the property of recognizing certain short DNA sequences and binding thereto with high affinity. The plasmid also contains a DNA sequence which is among the sequences recognized by P¹.

f) A reaction mixture is prepared containing the template prepared in e) and the oligonucleotide mixture obtained in d), at a concentration such that the ratio between the number of template molecules and the number of molecules of each mutant oligonucleotide is comprised between 0.01 and 100, preferably between 0.1 and 10. A thermostable polymerase is added together with all the necessary reagents to carry out replication of the template from the mutant oligonucleoties: reaction buffer, nucleotide triphosphates in sufficient amount, any required cofactors.

g) The mixture is subjected to an elevated temperature (greater than 80° C. and preferably approximately 94° C.) for at least one second and preferably for approximately one minute, so that single-stranded DNA will temporarily be present.

h) The temperature is lowered to a value comprised between 0 and 60° C. and preferably comprised between 20 and 50° C., for at least one second and preferably for approximately one minute, so that the oligonucleotides present in the mixture anneal to their site of homology in their target gene.

i) The reaction mixture is subjected to a temperature of approximately 68 to 72° C., which allows optimal activity of the polymerase, for a sufficient time to allow complete replication of the template, preferably the plasmid, and calculated according to the rate of synthesis of the polymerase (in bases per minute) and the size of the plasmid containing the target gene.

In step f), a thermostable ligase like Taq Ligase or Tth ligase can optionally be added. Advantageously in such case, the oligonucleotides will have been 5′ phosphorylated by means of a kinase prior to their use.

Steps g), h), and i) are repeated, preferably by using a thermocycler, so that the temperature cycles can be performed automatically.

j) Any suitable method is used to select the newly synthesized DNA strands generated in step i) from the initial templates.

k) The reaction mixtures obtained in j) are transformed into a suitable organism, such as yeasts or bacteria, rendered transformation-competent.

l) The cells transformed in step k) are transferred to a liquid culture, possibly with a suitable selection agent. Conditions (in particular: temperature) are used which allow expression of the protein of interest-P¹ fusion protein.

m) The templates, preferably the plasmids (genotype) are extracted, to which protein P¹ is bound and therefore indirectly the target protein (phenotype). Conditions are used (in particular: salt concentration) in which the bond between P¹ and the plasmid is conserved.

n) The actual selection is carried out: the complexes composed of the template-P¹-protein of interest are contacted with beads the surface of which is coated with ligand L¹ (alternatively, plates on which said ligand has been adsorbed are used). Plasmids encoding a protein having a high affinity for L¹ are bound to the beads; the other plasmids remain free in solution. The beads are isolated by centrifugation (alternatively, magnetic beads are used) and washed several times with a suitable medium.

o) The washed beads are recovered and placed in conditions (in particular: salt concentration) in which the bond between P¹ and the plasmid is no longer ensured. The mixture is centrifuged and the supernatant recovered. The DNA present in the supernatant is extracted (with a suitable kit or a known method, for example: phenol/chloroform extraction).

p) The series of steps g) to o) of the method according to this embodiment is repeated as many times as is necessary (between 0 and 100 times; generally 2 to 20 times). The templates recovered in step o) of one round of the protocol are used in step g) of the next round.

In a particular embodiment, the selection method used is not “plasmid display” but “cell-surface display” (Lee Sy et al., Trends Biotechnol. 2003 January; 21(1): 45-52). In such case, a suitable template, preferably a plasmid, is used in step e), the protein of interest is expressed as a fusion with a transport protein which anchors in the cytoplasmic membrane or the outer membrane of gram negative bacteria or in the wall of gram positive bacteria (U.S. Pat. No. 5,874,267, U.S. Pat. No. 6,274,345, U.S. Pat. No. 535,697), WO9324636, WO950479, WO9735022, WO9410330, WO9310214, WO9737025, WO9967366, WO0246388, WO006010, WO9709437, U.S. Pat. No. 5,616,686, WO9318163, U.S. Pat. No. 5,958,736, WO9640943 and U.S. Pat. No. 5,821,088). Alternatively, the protein of interest is expressed as a fusion with a transport protein which anchors in the wall of a yeast cell. In these cases, steps a) to d) are identical, step e) becomes:

‘e’) Independently, a template containing the target gene is prepared. The template, preferably a plasmid, also contains, flanking the target gene (upstream or downstream) and under control of the same promoter (so as to produce a fusion protein), the gene encoding a protein P² which has the property of being routed, in vivo, to the cell surface then bound to said surface, exposing the protein of interest on the outside of the cell.

Steps f) to k) are then identical and the subsequent steps are deleted and replaced by:

‘l’) The cells transformed in step k) are placed in liquid culture, possibly with a suitable selection agent. Conditions (in particular: temperature) are used which allow expression of the protein of interest-P² fusion protein at the cell surface.

‘m’) Using a suitable selection system (for example: coated microbeads, coated magnetic microbeads, FACS, microFACS), one isolates the subpopulation of cells which expose at their surface a protein of interest displaying a desired affinity for a ligand adapted to the property which one wants to improve. For example, and not by way of limitation, the ligand is an antigen, a substrate or a transition complex.

‘n’) Plasmid DNA is prepared from the selected cells. This DNA preparation is enriched in plasmids containing a gene encoding an improved protein. Said plasmids are transformed into bacteria, the transformed bacteria are cultured on solid medium containing a suitable selection agent, some or all of the individual clones obtained are cultured in liquid medium and each clone is subjected to a screening test. The plasmid DNA from clones considered improved in the screening test is sequenced. Alternatively, the plasmid DNA is prepared from the selected cells and steps f), g), h), i), j, k), l′), m′) and n′) of the method are repeated using this DNA instead of the plasmids prepared in e). In this way several successive rounds of molecular evolution by mutation-selection are performed.

In a particular embodiment, the method is readily adapted by those skilled in the art so that the selection method used is one of the following methods: “phage display” (Smith G P., Science 1985 228: 1315-1317; Gupta A et al., J. Mol. Biol. 2003 Nov. 21; 334(2): 241-54 and U.S. Pat. No. 6,593,081; U.S. 2003148372), “cell-surface display” (Kretzzschmar T et al., Curr. Opin. Biotechnol. 2002 December; 1 3(6): 598-602), compartmentalized self-replication (CSR; Ghadessy F H et al., Proc. Natl. Acad. Sci. USA 2001 Apr. 10; 98(8): 4552-7 and WO0222869), in vitro compartmentalization (Sepp A et al., FEBS Lett. 2002 Dec. 18; 532(3): 455-8 and WO9902671), “ribosome display” (Cesaro-Tadic S et al., Nat. Biotechnol. 2003 June; 21(6): 679-85; Matsuura T et al., FEBS Lett. 2003 Mar. 27; 539(1-3): 24-8; Amstutz P et al., J. Am. Chem. Soc. 2002 Aug. 14; 124(32): 9396-403 and U.S. Pat. No. 6,620,587; U.S. 2002076692), mRNA-peptide fusion or “mRNA display” (Nemoto N et al., FEBS Lett. 1997 Sep. 8; 414(2): 405-8; Takahashi, T. T et al., TIBS 28(3): 159-165).

### EMBODIMENT #7

### Mutagenesis and Selection of Nucleic Acids

In a seventh embodiment, the inventive method is characterized by molecular evolution of one or more different nucleic acids having novel or improved properties. The translation step is deleted and adaptations obvious to those skilled in the art are made. As an example, to evolve a catalytic RNA (a ribozyme), steps a) to j) can be carried out without modification (in which case the term gene of interest refers to a DNA complementary to the RNA of interest) and the following steps are replaced by: in vitro transcription, contact with the substrate and screening for RNA having a novel or improved catalytic activity.

### EMBODIMENT #8

### Case of Insertions/Deletions

In a particular embodiment, some or all of the oligonucleotides designed in step a), synthesized in step b) and placed in solution in the form of a mixture in step c) of any one of the embodiments described hereinabove do not introduce a substitution but an insertion or a deletion. In the case of a deletion, the oligonucleotides may, for example, be designed according to the following model:
5′-TTCATAGCTAGGCGGTGCATCC-3′ portion of target gene
3′-MGTATCG-CGCCACGTAGG-5′ oligonucleotide introducing a deletion

The oligonucleotide therefore has the following sequence:

3′-AAGTATCGCGCCACGTAGG-5′

and, at the end of the mutagenesis reaction, the three bases TAG are eliminated (deleted) and the gene therefore has the following sequence:

5′-TTCATAGCGCGGTGCATCC-3′.

In the case of an insertion, the oligonucleotides may, for example, be designed according to the following model:
5′-TTCATAGCTAG---GCGGTGCATCC-3′ portion of the target gene
3′-MGTATCGTAGCTTCGCCACGTAGG-5′ oligonucleotide introducing an insertion

Therefore the gene initially has the following sequence:

5′-TTCATAGCTAGGCGGTGCATCC-3′

and, at the end of the mutagenesis reaction, the three bases GAA are added (inserted) and the gene has the following sequence:

5′-TTCATAGCTAGGAAGCGGTGCATCC-3′.

### Embodiment #9

In a ninth embodiment, the inventive method is characterized by the following sequence of steps:

a) A mutagenesis strategy is designed in the same way as described in the first embodiment.

b) Based on this strategy, a set of mutant oligonucleotides is designed, preferably having a size comprised between 15 and 45 nucleotides and each homologous to a region of the target gene. In this particular embodiment, the outermost oligonucleotides have a reverse orientation, that is to say, each is homologous to a different strand of the target gene, so as to allow amplification of the DNA fragment located between said two oligonucleotides. The other oligonucleotides can be homologous to one or the other of the two strands indifferently.

c) The corresponding mutant oligonucleotides such as designed in step b) are synthesized using any type of chip-based method of synthesis.

Alternatively, a portion of the oligonucleotides, for example the two external oligonucleotides, can be synthesized by conventional chemical synthesis, whereas the other oligonucleotides are synthesized by using a DNA chip approach.

d) The oligonucleotides synthesized on the chip are released from their support, so as to obtain a mixture of oligonucleotides in solution.

e) Independently, a template containing the target gene is prepared.

f) A reaction mixture is prepared containing the template prepared in e) and the oligonucleotide mixture obtained in d), at a concentration such that the ratio between the number of template molecules and the number of molecules of each mutant oligonucleotide is comprised between 0.01 and 100, preferably between 0.1 and 10. A thermostable polymerase is added together with all the necessary reagents to carry out replication of the template from the mutant oligonucleoties: reaction buffer, nucleotide triphosphates in sufficient amount, any required cofactors.

g) The mixture is subjected to an elevated temperature (greater than 80° C. and preferably approximately 94° C.) for at least one second and preferably for approximately one minute, so that single-stranded DNA will temporarily be present.

h) The temperature is lowered to a value comprised between 0 and 60° C. and preferably comprised between 20 and 50° C., for at least one second and preferably for approximately one minute, so that the oligonucleotides present in the mixture anneal to their site of homology in their target gene.

i) The reaction mixture is subjected to a temperature of approximately 68 to 72° C., which allows optimal activity of the polymerase, for a sufficient time to allow complete replication of the plasmid, and calculated according to the rate of synthesis of the polymerase (in bases per minute) and the size of the target gene.

Steps g), h), and i) are repeated, preferably by using a thermocycler, so that the temperature cycles can be performed automatically.

j) Any suitable method is used to select the newly synthesized DNA strands generated in step i) from the starting templates.

j′) The DNA fragment synthesized in j) is used as an insert to be cloned in a previously linearized plasmid, for example by using the so-called “TA-cloning” approach, allowing rapid and efficient cloning of DNA fragments obtained by amplification.

k) The reaction mixture obtained in j′) is transformed into a suitable organism, such as yeasts or bacteria, rendered transformation-competent.

In step f), a thermostable ligase like Taq Ligase or Tth ligase can optionally be added. Advantageously in such case, the oligonucleotides will have been 5′ phosphorylated by means of a kinase prior to their use.

This embodiment of the invention can additionally contain one or more of steps 1),
m), n), o), or p) described hereinabove.

### Embodiment #10

In a tenth embodiment, the inventive method is characterized by the following sequence of steps:

Steps a), b), c), and d) are carried out in the same manner as in the first embodiment.

e) Independently, plasmid DNA is prepared from an ung− bacterial strain transformed by a plasmid containing the target gene. This plasmid DNA, being produced in an ung− strain, contains uracils instead of thymidines.

Steps f), g), h) and i) are carried out in the same way as described earlier. Step f) is carried out in the presence or absence of thermostable or thermosensitive ligase.

j) and k) To select newly synthesized DNA strands generated in step g) from the starting templates, one uses the selection system previously described by Kunkel et al. (Kunkel T A, Bebenek K, McClary J. Methods Enzymol. 1991; 204: 125-39): simply introducing the reaction mixture into ung+ bacteria (accounting for most laboratory strains, such as DH5a, DH10B, JM109, etc. . . . ) allows the selection of plasmids having been synthesized during steps f) to h), to the exclusion of the starting templates.

This embodiment of the invention can additionally contain one or more of steps l), m), n), o), or p) described hereinabove.

The invention also concerns a method of mutagenesis of a target protein or of several target proteins, characterized in that it comprises preparing a mutant gene expression library from a target gene coding for said protein, or from several target genes coding for said proteins, according to the mutagenesis method described hereinabove, then expressing said mutant genes to produce a library of mutant proteins, and optionally screening said mutant proteins for a desired function, advantageously by comparison with the target protein.

The invention also has as its object a mixture containing mutant oligonucleotides of one or more target gene(s), having been produced such as described in steps a), b), c), and d) hereinabove. In a particular embodiment, the mixture contains all oligonucleotides sufficient to generate all possible substitutions in one or more target genes, i.e. a number of oligonucleotides equal to nineteen times the number of codons encoded by said target gene(s). In a second particular embodiment, the mixture contains all oligonucleotides sufficient to generate alanine substitution of each codon in one or more target genes, i.e., as many oligonucleotides as there are codons in said target gene(s), after deducting codons already encoding an alanine.

The invention further has as its object a mutant gene library that can be obtained by one of the methods described hereinabove.

Other advantages and characteristics of the invention will become apparent in the following examples, which are not given by way of limitation, as well as in the appended drawings.

### LEGENDS OF FIGURES

FIG. 1. Alignment of clones sequences obtained in Example 8.

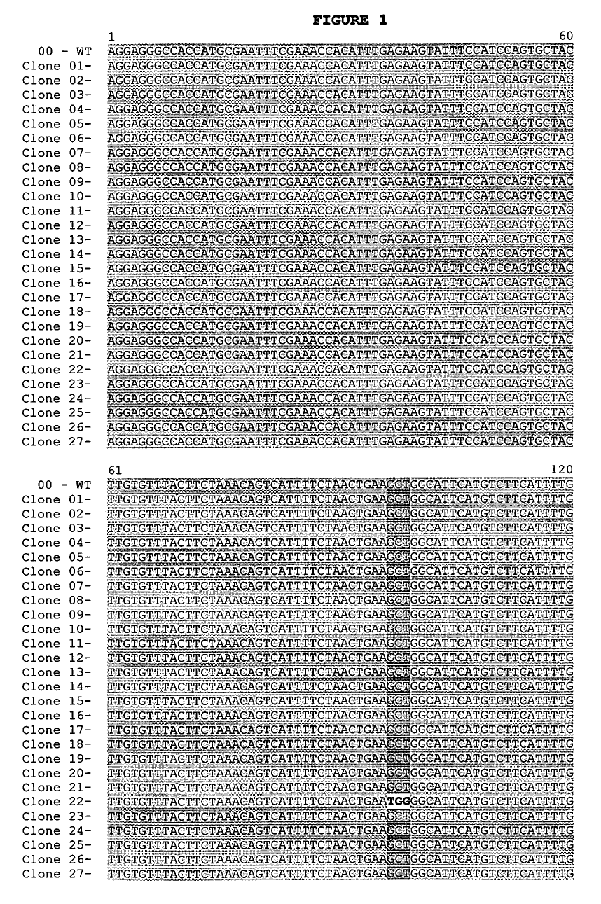

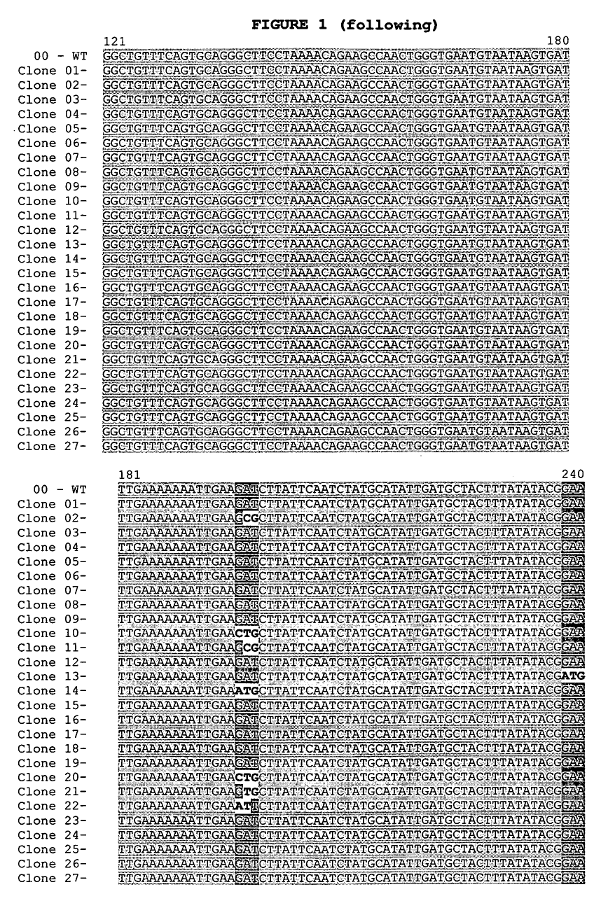

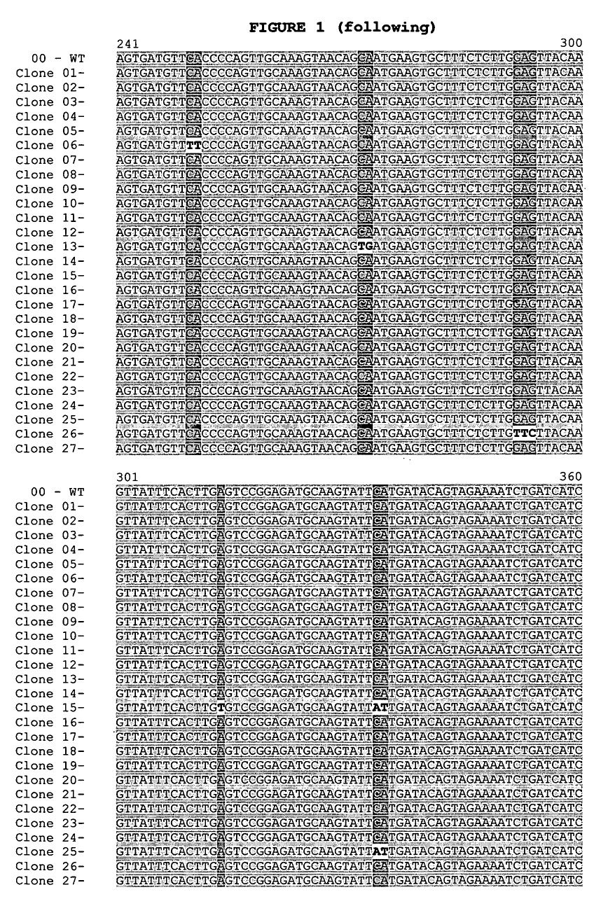

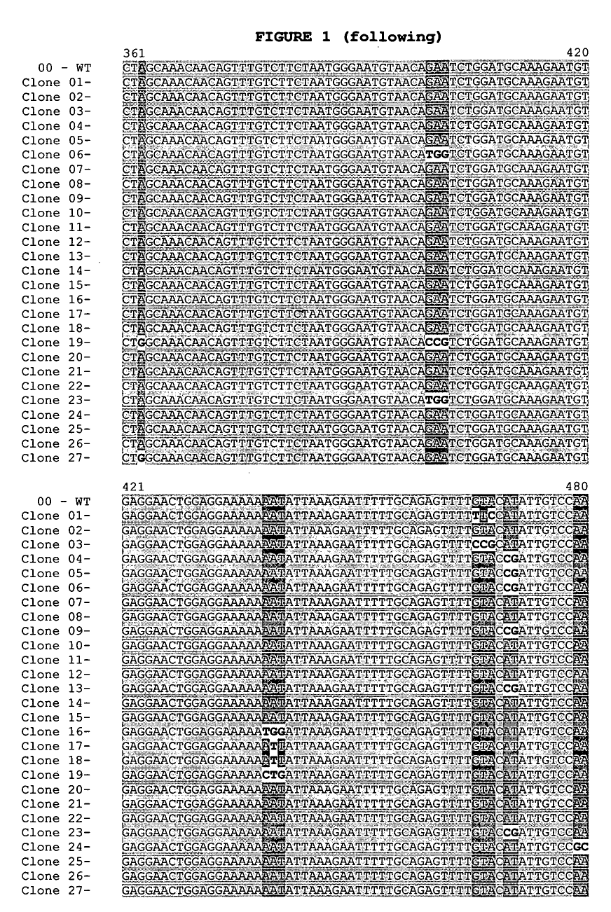

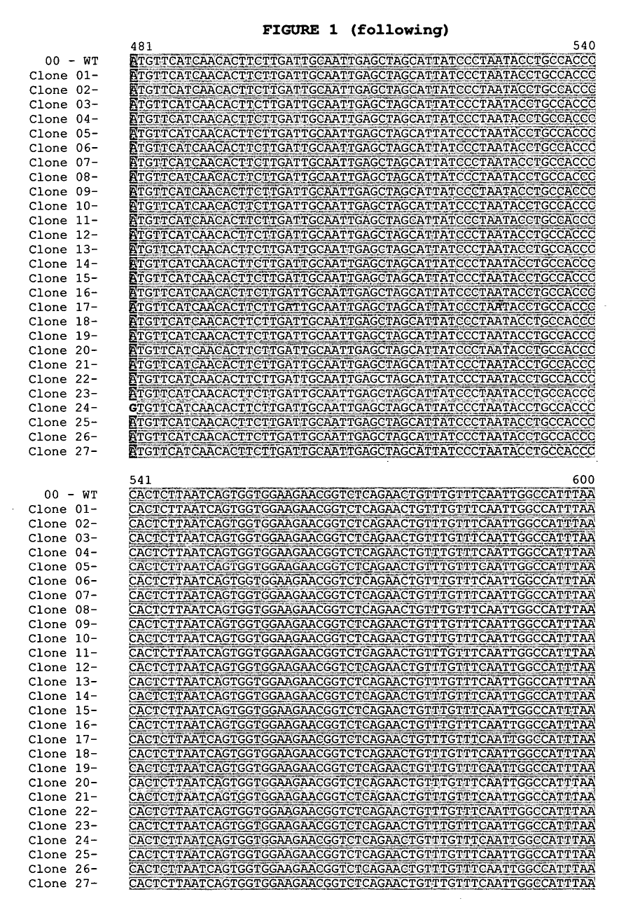

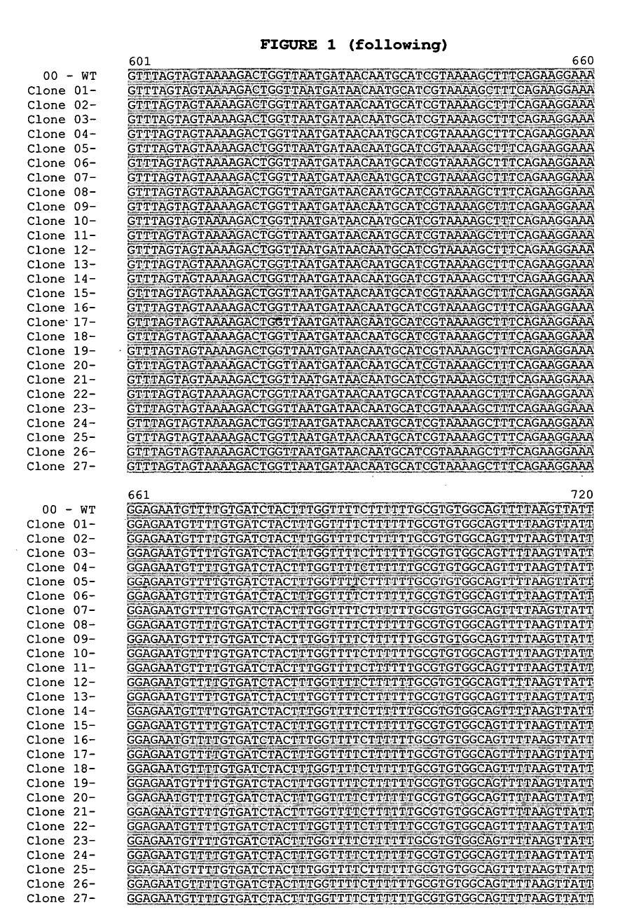

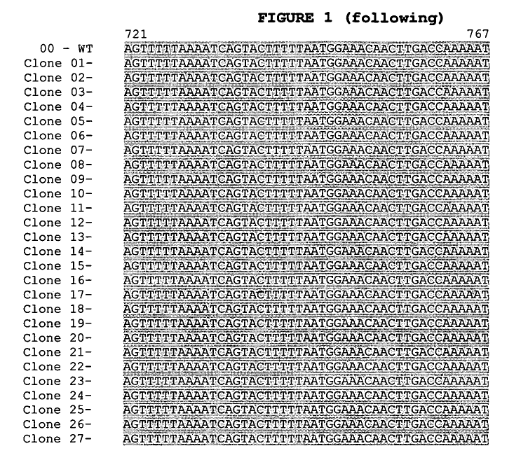

### EXAMPLES

### Example 1

### Molecular Evolution of an Amylase

The amylases are a family of enzymes which act on starch, cleaving it into smaller carbohydrate chains or even monomers. Amylases are used in many fields of industry, and in particular in the food processing industry and in detergents.

Here, the objective was to improve the activity of an amylase in conditions of low starch concentration, by lowering its Km.

a) A mutagenesis strategy by which to lower the Km of this amylase was designed. Since the amylases are extremely well characterized and several of these enzymes have been crystallized (x-ray resolution of their structure), a mutagenesis strategy was designed on the basis of these structures. More specifically, the active site residues as well as direct neighboring residues were targeted. In all, then, about thirty residues were targeted. The aim was to produce a combinatorial substitution, with an average of two mutations per molecule, not with the entire possible diversity, but only with structurally similar residues, i.e., belonging to the same subclass of amino acids (hydrophobic, aromatic, etc.), which represents an average of 5 substitutions per target residue.

b) Based on this strategy, a set of mutant oligonucleotides was designed in which the mutant codon was flanked on either side by 15 bases perfectly homologous to the target sequence. Approximately 150 oligonucleotide 33mers were therefore designed (30 target residues multiplied by an average of 5 substitutions).

c) The mutant oligonucleotides such as designed in b) were synthesized by using a chip-based method of oligonucleotide synthesis. Beforehand, a thin layer of compound A, a basolabile compound, was adsorbed on the chip. The maximum number of oligonucleotides to be synthesized on such chip is approximately 8000: each of the 150 oligonucleotides was thus synthesized in several copies (approximately 50) so as to have a large amount of the oligonucleotide mixture.

d) The oligonucleotides were released from their support, to be put in solution. To do this, the chip was placed in basic conditions, the effect of which is to cleave the basolabile spacer and automatically release the oligonucleotides into suspension. Before using them, the oligonucleotides were dried by evaporation of ammonia under low pressure, then resuspended either in water or a suitable buffer. The concentration of said oligonucleotides was determined by the classical spectrophotometric approach.

e) Separately, a plasmid DNA miniprep was prepared from bacteria transformed by the plasmid containing the amylase target gene, a bacterial promoter driving expression of said gene, and the ampicillin resistance gene.

f) A reaction mixture in a volume of 7.5 microliters was prepared containing 100 nanograms of template prepared in e) and 5 picomoles of the oligonucleotide mixture obtained in d).

g) The reaction mixture was subjected to a temperature of 95° C., so that single-stranded DNA would temporarily be present.

h) The tube containing the mixture was allowed to cool to room temperature, so that the oligonucleotides present in the mixture would anneal to their site of homology in the target gene.

i) 0.5 μl of T4 polymerase (New England Biolabs) was added, together with 1 microliter of its 10× buffer and 1 microliter of a solution containing the four deoxyribonucleotide triphosphates at a total concentration of 1 mM. The reaction mixture was incubated at a temperature of 37° C. for 20 minutes.

j) 0.5 μl of Dpn I enzyme (New England Biolabs), 2 microliters of NEB 4 buffer and 7.5 microliters of distilled water were added, and the reaction mixture was incubated at 37° C. for 30 minutes, so that the starting templates would be cleaved.

k) The reaction mixture obtained in j) was transformed into competent bacteria, using the heat shock transformation technique.

l) The transformed bacteria were plated on a large format petri dish containing, in addition to the required nutritive media (LB), a sufficient amount of ampicillin. The next day, about 10,000 bacterial colonies were obtained, each containing a different, possibly mutant plasmid.

The mutational content of these colonies was studied by sequencing a statistical sample. As long as the observed mutation rate was not sufficient, i.e., as long as it was less than 2 mutations per molecule on average, DNA was prepared from the bacterial colonies obtained in l), and steps f) to l) were repeated for as many times as necessary to achieve the desired mutation rate. To increase the efficiency of the reaction and therefore minimize the number of rounds of steps f) to l), 0.5 μl of T4 ligase and 1 μl of its 10× buffer can be added at step l). In such case, the oligonucleotides obtained in d) have to be phosphorylated by means of a kinase (PNK, New England Biolabs for example) prior to their use in step f). After 2 to 4 rounds, the rate of mutagenesis was greater than 2.

m) The bacterial colonies were individually isolated and inoculated into a nutritive medium containing ampicillin, either by manual subculturing, or by using special colony subculturing robotic equipment.

n) The activity of the protein obtained after lysing the cultures obtained in m) was measured and said activity was compared with that of the protein produced under the same conditions from plasmid DNA containing the non-mutant target gene. These activity measurements can be carried out using one of the classical tests of amylase activity, such as the iodine test (Guan, H. P. and Preiss, J. (1993) Plant Physiol. 102: 1269-1273), or the “reducing sugars” test (M Lever (1973) Biochemical Medicine 7: 274-281). When the activity associated with a bacterial colony was reproducibly found to be significantly higher than that observed using the target gene, the mutant molecule was studied more thoroughly, first by enzymatic tests to determine its Km, and then by sequencing the mutant gene, so as to identify the nature of the mutation underlying said improved activity.

### Example 2

### Alanine Scanning of an Acylase

The acylases are enzymes used in many industrial fields, and particularly in the field of beta-lactam antibiotic synthesis. Many studies characterizing the activity of these enzymes have been carried out, but the precise mechanism by which they function has still not been fully elucidated. It was therefore helpful to carry out a complete Alanine Scan on one of these enzymes, so as to establish a complete functional map. The objective was to generate all the alanine mutants of this enzyme, and to test all these mutants by means of a simple functional test. Mutants having lost their activity were then sequenced to identify the position or positions underlying said loss of activity. A parallel approach was used here, in which all the mutants were generated in the same reaction; the desired mutation rate was less than one mutation per molecule on average, so as to have mainly point mutants.

a) The mutagenesis strategy consisted of targeting all of the residues of the acylase useful for the antibiotic synthesis, with the exception of the first (translation initiation codon), the last (translation termination codon) and all the codons naturally associated with an alanine. In all, approximately 700 positions were targeted.

b) The 700 mutant oligonucleotides were designed. In each of these 33-mer oligonucleotides, the mutant codon was flanked on either side by 15 bases perfectly homologous to the corresponding region in the target gene. The mutant codon was invariably of the type GCG, because this codon is most favorable for expression in E. coli.

c) The mutant oligonucleotides such as designed in b) were synthesized using a chip-based method of oligonucleotide synthesis. Beforehand, a thin layer of compound A, a basolabile compound, was adsorbed on the chip. The maximum number of oligonucleotides to be synthesized on such chip is approximately 8000: each of the 700 oligonucleotides was thus synthesized in several copies (approximately 10) so as to have a large amount of the oligonucleotide mixture.

d) The oligonucleotides were released from their support, to be put in solution. To do this, the chip was placed in basic conditions, the effect of which is to cleave the basolabile spacer and automatically release the oligonucleotides into suspension. The concentration of said oligonucleotides was determined by the classical spectrophotometric approach.

e) Separately, a plasmid DNA miniprep was prepared from bacteria transformed by the plasmid containing the acylase gene, a bacterial promoter driving expression of said gene, and the tetracycline resistance gene.

f) The following reaction mixture was prepared:
200 nanograms of template prepared in e)
10 picomoles of the oligonucleotide mixture obtained in d)
1 microliter of a mixture of the four deoxyribonucleotide triphosphates at a total concentration of 100 mM
2.5 μl of Pfu polymerase 10× buffer
0.5 μl of Pfu Polymerase
1 μl of 100 mM MgSO4
complete with distilled water to 25 μl

g) The reaction mixture was subjected to a temperature of 94° C., so that single-stranded DNA would temporarily be present.

h) The reaction mixture was subjected to a temperature of 45° C., so that each oligonucleotide present in the mixture would anneal to its site of homology in the target gene.

i) The reaction mixture was subjected to a temperature of 68° C. for 20 minutes, a time sufficient for the entire plasmid to be replicated.

Steps g), h), and i) were repeated 11 times, using a thermocycler to automatically perform the temperature cycles.

j) To 10 μl of the previous reaction mixture were added 0.5 μl of enzyme Dpn I (New England Biolabs), two microliters of NEB 4 buffer and 7.5 microliters of distilled water. The reaction mixture was incubated at 37° C. for 30 minutes so that the starting templates would be cleaved.

k) The reaction mixture obtained in j) was transformed into competent bacteria, using the heat shock transformation technique.

l) The transformed bacteria were plated on a large format petri dish containing, in addition to the required nutritive media (LB), a sufficient amount of tetracycline. The next day, about 10,000 bacterial colonies were obtained, each containing a different, possibly mutant plasmid.

The mutational content of these colonies was studied by sequencing a statistical sample. As long as the observed mutation rate was not sufficient, i.e., as long as it was less than 0.8 mutations per molecule on average, DNA was prepared from the bacterial colonies obtained in l), and steps f) to l) were repeated for as many times as necessary to achieve the desired mutation rate. To increase the efficiency of the reaction and therefore minimize the number of rounds of steps f) to l), 0.5 μl of Pfu ligase and 1.25 μl of its 10× buffer can be added at step 1), replacing half of the Pfu polymerase 10× buffer. In such case, the oligonucleotides obtained in d) have to be phosphorylated by using a kinase (PNK, New England Biolabs for example) and ATP, prior to their use in step f). After 1 to 3 rounds, the rate of mutagenesis was greater than 0.8.

m) The bacterial colonies obtained were isolated individually and inoculated into nutritive medium containing tetracycline, either by manual subculture, or by using special colony subculturing robotic equipment.

n) The activity of the protein obtained after lysing the cultures obtained in m) was measured and said activity was compared with that of the protein produced under the same conditions from plasmid DNA containing the non-mutant target gene. These activity measurements can be carried out using one of the classical tests of acylase activity, such as the test based on fluoram derivation of the reaction product. When the activity associated with a bacterial colony was reproducibly found to be significantly higher than that observed using the target gene, the mutant molecule was studied more thoroughly, first by enzymatic tests to determine its enzymatic parameters, and then by sequencing the mutant gene, so as to identify the mutation underlying said improved activity and to thereby complete the functional map being elaborated for said enzyme.

### Example 3

### Stabilization of Gamma Inteferon

In this example, the aim was to generate an improved mutant of gamma interferon, used in the treatment of hepatitis.

The objective was to obtain a molecule that is more stable, so as to decrease the number of injections to one a week instead of three per week with gamma interferon having the natural sequence. a) A mutagenesis strategy was designed: Even though the structure of gamma interferon and its interactions with other molecules have been characterized in detail, designing a strategy to improve its stability is not straightforward. Therefore, to maximize the chances of obtaining a positive mutant, a good strategy to pursue consisted of targeting all the residues (165) and introducing at each residue the maximum diversity (the 19 possible residues), all in a combinatorial approach, with an average of 2 mutations per molecule.

b) (165×19) or 3135 mutant oligonucleotides were designed. In each oligonucleotide, the mutant codon was flanked on either side by 15 bases perfectly homologous to the corresponding region of the target gene.

c) The mutant oligonucleotides such as designed in b) were synthesized by using a chip-based method of oligonucleotide synthesis. Beforehand, a thin layer of compound A, a basolabile compound, was adsorbed on the chip. The maximum number of oligonucleotides to be synthesized on such chip is approximately 8000: each of the 3135 oligonucleotides was thus synthesized in two copies so as to have a large amount of the oligonucleotide mixture.

d) The oligonucleotides were released from their support, to be put in solution. To do this, the chip was placed in basic conditions, the effect of which is to cleave the basolabile spacer and automatically release the oligonucleotides into suspension. The concentration of said oligonucleotides was determined by the classical spectrophotometric approach.

e) Separately, a plasmid DNA miniprep was prepared from bacteria transformed by the plasmid containing the gamma interferon gene, a eukaryotic promoter driving expression of said gene, and the ampicillin resistance gene under control of a bacterial promoter.

f) The following reaction mixture was prepared:
200 nanograms of template prepared in e)
10 picomoles of the oligonucleotide mixture obtained in d)
1 microliter of a mixture of the four deoxyribonucleotide triphosphates at a total concentration of 100 mM
2.5 μl of Pfu polymerase 10× buffer
0.5 μl of Pfu Polymerase
1 μl of 100 mM MgSO4
complete with distilled water to 25 μl

g) The reaction mixture was subjected to a temperature of 94° C., so that single-stranded DNA would temporarily be present.

h) The reaction mixture was subjected to a temperature of 45° C., so that each oligonucleotide present in the mixture would anneal to its site of homology in the target gene.

i) The reaction mixture was subjected to a temperature of 68° C. for 20 minutes, a time sufficient for the entire plasmid to be replicated.

Steps g), h), and i) were repeated 11 times, using a thermocycler to automatically perform the temperature cycles.

j) To 10 μl of the previous reaction mixture were added 0.5 μl of enzyme Dpn I (New England Biolabs), two microliters of NEB 4 buffer and 7.5 microliters of distilled water. The reaction mixture was incubated at 37° C. for 30 minutes so that the starting templates would be cleaved.

k) The reaction mixture obtained in j) was transformed into competent bacteria, using the heat shock transformation technique.

l) The transformed bacteria were plated on a large format petri dish containing, in addition to the required nutritive media (LB), a sufficient amount of ampicillin. The next day, about 10,000 bacterial colonies were obtained, each containing a different, possibly mutant plasmid.

The mutational content of these colonies was studied by sequencing a statistical sample. As long as the observed mutation rate was not sufficient, i.e., as long as it was less than 2 mutations per molecule on average, DNA was prepared from the bacterial colonies obtained in l), and steps f) to l) were repeated for as many times as necessary to achieve the desired mutation rate. To increase the efficiency of the reaction and therefore minimize the number of rounds of steps f) to l), 0.5 μl of Pfu liganse and 1.25 μl of its 10× buffer can be added at step 1), replacing half of the Pfu polymerase 10× buffer. In such case, the oligonucleotides obtained in d) have to be phosphorylated by using a kinase (PNK, New England Biolabs for example) and ATP, prior to their use in step f). After 2 to 4 rounds, the rate of mutagenesis was greater than 2.

m) The bacterial colonies obtained were individually isolated and inoculated into nutritive medium containing ampicillin, either by manual subculture, or by using special colony subculturing robotic equipment. Each colony contains a target gene potentially mutated at one or more sites, integrated in the plasmid.

n) Plasmid DNA was prepared from each of the cultures prepared in m).

o) The plasmid DNA preparation obtained in n) was used to express the corresponding protein. To to this, each set of plasmid DNA was separately introduced into mammalian cells, by transfection.

p) The activity of each gamma interferon mutant obtained was measured in the supernatant of cells transfected in o), and said activity was compared with that of the protein produced under the same conditions from plasmid DNA containing the non-mutant target gene. These activity measurements can be carried out using one of the classical tests of gamma interferon activity. To measure stability of the mutants, it was necessary to preincubate the mutant molecules, at a given temperature, so as to measure the decrease in activity in these conditions, and compare this decrease with that observed for the non-mutant gene. When the decrease in activity for a particular clone was lower than that seen with the non-mutant gene, all necessary measures were taken to characterize the gain in stability, and the mutant gene was sequenced so as to identify the nature of the mutation underlying the improved activity.

### Example 4

### Massive Multiplex Mutagenesis: Use of a Single Oligonucleotide Mixture Synthesized on a Chip for Alanine Scanning of Several Genes Simultaneously

For additional savings, massive mutagenesis strategies can be carried out on several genes simultaneously, by using a single oligonucleotide mixture. This example describes the complete alanine scanning of four genes simultaneously, although this approach can be adapted for any mutagenesis strategy.

a) A mutagenesis strategy was designed for several target genes. In each target gene, all codons except the first and last codon and the codons naturally associated with an alanine were targeted. Care was taken to choose target genes that did not share too much homology, so that the oligonucleotides intended to mutate one of the genes would not hybridize to another, which could introduce additional unwanted mutations.

b) Based on this strategy, a set of mutant oligonucleotides was designed. The oligonucleotides here are analogous to those described in example 2.

c) The mutant oligonucleotides such as designed in b) were synthesized by using a chip-based method of oligonucleotide synthesis, as described in the previous examples. All the oligonucleotides, intended for each of the four genes, were synthesized on a single chip.

d) The oligonucleotides were released from their support, to be put in solution.

e) Separately, the four plasmids each containing a target gene were prepared so as to provide a sufficient amount of purified plasmid DNA. In addition to the target gene and associated promoter sequence, each plasmid contained a different antibiotic resistance gene (for example, the first plasmid contained the ampicillin resistance gene, the second the kanamycin resistance gene, the third the tetracycline resistance gene and the fourth the chloramphenicol resistance gene).

f) A reaction mixture was prepared containing 100 ng of each template prepared in e) and the oligonucleotide mixture obtained in d). The reagents described in example 2 were then added to the reaction mixture.

Steps g) to k) were carried out as described in example 2.

l) The bacteria transformed with the reaction mixture were divided into four fractions, each of which was plated on a medium containing a different selection agent. Thus, only those bacteria containing the first plasmid will grow on petri dishes containing ampicillin, while bacteria containing mutants of the second plasmid will grow on a petri dish containing kanamycin, and so forth. In this manner, the mutant libraries corresponding to each plasmid were separated into four sub-libraries each containing the mutants corresponding to a single target gene.

The remainder of this example is identical to that described in example 2, apart from the fact that the several rounds of steps f) to l) included an additional step of combining the DNA obtained from each of the four sub-libraries.

### Example 5

### Second Example of Massive Multiplex Mutagenesis

This example is similar to the previous one but allows the concurrent use of six different plasmids, and isolation of their corresponding mutant libraries, at the end of the experiment, by means of a simple selection on selective media.

As just four main antibiotics are commonly used in research studies, it is only by using combinations of these antibiotics and combinations of antibiotic resistance genes that one can simultaneously use this many plasmids and easily re-isolate them.

Thus, each of the six templates contained, in addition to the target gene, two resistance genes (Amp-Kan; Amp-Tet; Amp-Cam; Tet-Kan; Tet-Cam; Kan-Cam).

The protocol was performed as in example 4. At the end of the protocol, the transformed bacteria were plated on selective media containing two antibiotics, so as to isolate each of the six resulting mutant sub-libraries.

### Example 6

### Use of a Single Oligonucleotide Mixture Synthesized on a Chip to Carry Out Mutagenesis Strategies on Several Genes Sequentially

This example is similar to example 4 but allows a theoretically infinite number of different plasmids to be used, each containing a different target gene. In this example, the plasmids were not used all at the same time, but sequentially, thereby avoiding the aforementioned problem of isolating the mutant sub-libraries: a single oligonucleotide mixture was synthesized as in the previous examples, and said mixture was then used in several independent reactions each containing a different target gene. The remainder of the method was then analogous to that described in example 2.

### Example 7

### Simultaneous Creation of Mutants in Two Target Genes

In some cases, it may be necessary to creat mutant libraries for two genes simultaneously, and to simultaneously screen the two mutant gene libraries.

This requirement applies in particular when the genes have a synergistic effect. A specific example is that of two subunits of a same protein. Other cases, such as the case of vaccines in particular, can also reveal strong synergies between two genes. In all these cases, the simultaneous creation of mutant libraries of two genes, and the co-expression of these two types of mutant molecules, can be a part of a global molecular evolution strategy.

Here the starting plasmid contained not one but two target genes, each under control of a eukaryotic or prokaryotic promoter, according to the study model. (Having both target genes cloned in the same plasmid simplifies the subsequent steps of transformation or transfection. However, it is also possible to use two plasmids each containing one target gene).

An oligonucleotide mixture was synthesized as described in the previous examples: some oligonucleotides in the mixture were designed to introduce mutations in the first gene, the others in the second.

This oligonucleotide mixture was then used to generate a mutant plasmid library, which for example can be synthesized and used as in example 4.

### Example 8

### Use of an Oligonucleotide Mixture Synthesized on a Chip for Mutagenesis of IL15

The model used in this example is the IL15 (interleukin 15) gene cloned in the pORF vector (IL15 sequence SEQ ID No 1). The 296 oligonucleotides modify 37 sites in the IL15 gene, 18 of which correspond to elimination of restriction sites (oligonucleotide sequences and modified sites: SEQ ID Nos 3-298). The others concern positions 157, 490, 205, 238, 265, 292, 175, 226, 250, 280, 301, 325, 346, 370, 391, 415, 433, 457, and 478 of the IL15 gene. Each site was mutagenized by the following codons: GCG; TTC; ATT; CTG; CCG; GTG; TGG; ATG.

The 18 restrictions sites are as follows:

| Modified | Position of the mutation | |
|---|---|---|
| restriction site | in Seq ID No 1 | SEQ ID Nos |
| MslI | 220-222 | 3-10 |
| XmnI | 37-39 | 11-18 |
| AluI | 97-99 | 19-26 |
| BsmI | 100-102 | 27-34 |
| BglII | 196-198 | 35-42 |
| SmlI | 310-312 | 43-50 |
| NsiI | 214-216 | 51-58 |
| BsrDI | 271-273 | 59-66 |
| BspHI | 334-336 | 67-74 |
| BfaI | 361-363 | 75-82 |
| SspI | 439-441 | 83-90 |
| RsaI | 466-468 | 91-98 |
| BsrGI | 469-471 | 99-106 |
| BsaWI | 316-318 | 107-114 |
| TfiI | 400-402 | 115-122 |
| MseI | 445-447 | 123-130 |
| MlyI | 313-315 | 131-138 |
| TaqI | 22-24 | 139-146 |
| — | 157-159 | 147-154 |
| — | 490-492 | 155-162 |
| — | 205-207 | 156-170 |
| — | 238-240 | 171-178 |
| — | 265-267 | 179-186 |
| — | 292-294 | 187-194 |
| — | 175-177 | 195-202 |
| — | 226-228 | 203-210 |
| — | 250-252 | 211-218 |
| — | 280-282 | 219-226 |
| — | 301-303 | 227-234 |
| — | 325-327 | 235-242 |
| — | 346-348 | 243-250 |
| — | 370-372 | 251-258 |
| — | 391-393 | 259-266 |
| — | 415-417 | 267-274 |
| — | 433-435 | 275-282 |
| — | 457-459 | 283-290 |
| — | 478-480 | 291-298 |

The oligonucleotides were synthesized on a support of porous silica to which they were coupled via a cleavable spacer. The method to functionalize a support with such spacer is described for example in WO03008360. The synthetic method is described in WO0226373. More particularly, the cleavable spacer is a t-butyl-11-(dimethylaminodimethylsilyl)undecanoate which is bonded on the silica support and the ester group of which is deprotected.

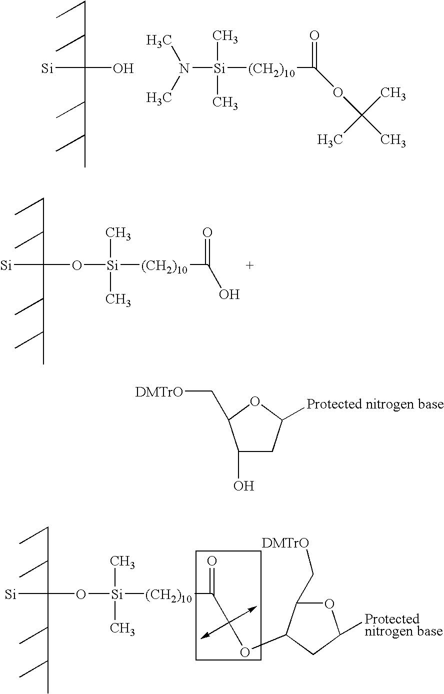

The oligonucleotides were synthesized on a solid support according to the method described in WO0226373 (the teachings thereof being incorporated as reference). They were synthesized on 4 chips: 1 chips for oligonucleotides having an A in 3′, 1 for a C in 3′, 1 for a G in 3′, and 1 for a T in 3′. The oligonucleotides were then released by treatment at basic pH in ammonia solution.

This synthesis yielded a pool of 296 oligonucleotides in a volume of 10 μl, at a total concentration of 30 pmol for the whole of the 296 oligonucleotides. The mixture of oligonucleotides was then used to generate a mutant library according to the following protocol.

1—Oligonucleotide Purification
Dilute 3 μl or 2 μl of the oligonucleotide mixture in 100 μl of H₂O;
Load on a Centricon YM3 column (Millipore; centrifuge for 40 min. at 9000 rpm; and
Invert the column and recover 15 μl after centrifuging for 1 min at 9000 rpm.

2—Phosphorylation of the Oligonucleotides

```
15 μl of purified oligonucleotides
  2 μl of PNK buffer; 2 μl of 10 mM ATP
  1 μl of PNK
V_{f} = 20 μl
1 h at 37° C.; no inactivation at 65° C.
```

3—PLCR (Polymerase Ligase Chain Reaction)

| | |
|---|---|
| 1 μl (200 ng of pORF IL15 template) | 1 μl of 10 mM ATP |
| 1 μl of dNTP (25 mM) | 0.2 μl of NAD (100 mM) |
| 1 μl of MgSO₄ (100 mM) | 0.2 μl of dTT (1 M) |
| 3.5 μl of pfu pol 10× buffer | 0.8 μl of pfu pol |
| 0.8 μl of Tth ligase | 0.5 μl of Taq |

V_{f} = 10 μl

Reaction: 10 μl of mix+20 μl of phosphorylated oligonucleotides+5 μl of H₂O
Negative control: 10 μl of mix+25 μl of H₂O
Thermocycler program: 1′ at 94° C.; 2′ at 40° C.; 20′ at 68° C.; 12 cycles

4—Dpn I Digestion
35 μl of PLCR
4 μl of buffer 4 (NEB); 0.5 μl of Dpn I (20,000 U/mL)
0.5 μl of H₂O 30′ at 37° C.

5—Dialysis+Transformation
8 μl dialysed on membrane against H₂O for 30′; electroporation with 40 μl into electrocompetent DH10B bacteria; take up in 1 mL of SOC, then 45′ at 37° C.
Centrifuge for 4′ at 6000 rpm, then take up in 200 μl of LB
Plate on LB+Amp. medium: 5000 colonies (dish No. 1: 9/10)
Subculture 96 colonies on dish in LB+Amp. medium and grow in shaker culture at 37° C. for 3 hours: 3 dishes

6—PCR on Cultures
Mix for 96 reactions:
4.5 mL of H₂O; 500 μl of thermopol buffer; 100 μl of dNTP (2.5 mM)
20 μl of IL15 oligo (421) (100 μM); 20 μl of IL15 oligo (1500) (100 μM)
250 μl of Taq; 50 μl of mix+5 μl of culture
Thermocycler program: 10′ at 96° C.; [1′ at 94° C.; 1′ at 50° C.; 1′30 at 72° C.]; 35 cycles

7—Digestion of PCR Products
Digestion was done in 96-well plates (1 unit per well)
10 μl of PCR+10 μl of MIX; restriction enzymes: BsrG I; Bgl II; Ssp I; Mly I

8—Sequencing of the Clones

9—Harvesting of Libraries+Additional Rounds of PLCR

Colony dish No. 1 (see step 5) was harvested and DNA was then prepared (E.Z.N.A™ Plasmid Miniprep kit). 200 ng of this library (No. 1) served as template for a second round of PLCR with 2 μl of the purified oligonucleotide pool (see steps 1 and 2). This library (No. 2) was screened for mutants according to the previously described protocol.

After library (No. 2) was harvested, 200 ng of the DNA preparation was used to carry out a third round of PLCR, with another 2 μl of the purified oligonucleotide pool. This library (No. 3) from the third round of PLCR was screened for mutants.

Results

Selection of clones was based on loss of a restriction enzyme site in the IL15 gene. The following restriction enzymes were used for this screen: BsRG I, Bgl II, Ssp I, and Mly I. Seven other randomly selected clones (noted *) were added, without any prior analysis of the restriction profile.

| CLONE | Round of PLCR | MUTATION |
|---|---|---|
| 1 | 1^{st} | BsRG I |
| 2 | 1^{st} | Bgl II |
| 3 | 1^{st} | BsrG I |
| 4 | 2^{nd} | BsrG I |
| 5 | 2^{nd} | BsrG I |
| 6 | 2^{nd} | position 251/Tfi I/BsrG I |
| 7 | 2^{nd} | WT IL15* |
| 8 | 2^{nd} | WT IL15* |
| 9 | 2^{nd} | BsrG I |
| 10 | 2^{nd} | Bgl II |
| 11 | 2^{nd} | Bgl II |
| 12 | 3^{rd} | WT IL15* |
| 13 | 3^{rd} | position 237/BsrG I/BsrD I |
| 14 | 3^{rd} | Bgl II |
| 15 | 3^{rd} | BspH I/Mly I |
| 16 | 3^{rd} | Ssp I |
| 17 | 3^{rd} | Ssp I |
| 18 | 3^{rd} | Ssp I |
| 19 | 3^{rd} | Bfa I/Tfi I/Ssp I |
| 20 | 3^{rd} | Bgl II |
| 21 | 3^{rd} | Bgl II |
| 22 | 3^{rd} | Alu I/Bgl II |
| 23 | 3^{rd} | Tfi I/BsrG I |
| 24 | 3^{rd} | position 478* |
| 25 | 3^{rd} | BspH I* |
| 26 | 3^{rd} | position 292* |
| 27 | 3^{rd} | Bfa I* |

*randomly selected clones

This example demonstrates for the first time that mutants can be prepared from an oligonucleotide library synthesized on a DNA chip. The quality of the oligonucleotide array synthesized on the chip is sufficient and the quality is comparable to the one obtained with classical synthesis.

1- A method for producing a library of mutant genes comprising the following steps:
a. Synthesizing on a solid support an oligonucleotide library comprising oligonucleotides complementary to one or more regions of one or more target genes and each comprising, preferably in their center, one or more mutations of the sequence of the target gene(s);
b. Placing the oligonucleotide library obtained in a) in solution; and,
c. Generating a library of mutant genes by using the oligonucleotide library in solution obtained in b) and one or more templates containing said target gene(s).

2- The method according to claim 1, wherein the mutant gene library is generated in step c) by a Massive Mutagenesis method.

3- The method according to claim 1, wherein step c) comprises the following steps:
i. Providing one or more templates containing said target gene(s);
ii. Contacting said template(s) with the oligonucleotide library synthesized in a) in conditions that allow the oligonucleotides in the library to anneal to said template(s) so as to produce a reaction mixture;
iii. Carrying out a replication of said template(s) from the reaction mixture by using a DNA polymerase;
iv. Eliminating the starting template(s) from the product of step iii) and thereby selecting newly synthesized DNA strands; and, optionally,
v Transforming an organism with the DNA mixture obtained in step iv).

4- The method according to claim 1, wherein the template is a circular nucleic acid, preferably a plasmid.

5- The method according to claim 1, wherein the template contains elements enabling the expression of said target gene(s).

6- The method according to claim 1, wherein the oligonucleotides of said library synthesized on the solid support are coupled to said solid support via a cleavable spacer molecule and wherein said oligonucleotides are placed in solution by subjecting the oligonucleotides coupled to said solid support to conditions associated with cleavage of the spacer molecule.

7- The method according to claim 6, wherein said spacer molecule can be cleaved in basic medium, by reaction to light or by enzymatic reaction.

8- The method according to claim 7, wherein said spacer molecule can be cleaved in basic medium.

9- The method according to claim 8, wherein said spacer molecule is the compound represented by the formula:

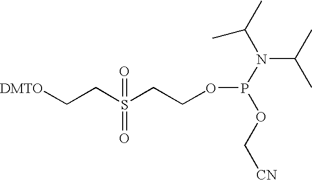

10- The method according to claim 8, wherein said spacer molecule is the compound represented by the formula:

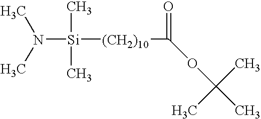

11- The method according to claim 3, in which step iv) is carried out by means of a restriction enzyme specific for methylated DNA strands, preferably belonging to the group of enzymes: DpnI, NanII, NmuDI and NmuEI.

12- The method according to claim 1, wherein the oligonucleotides synthesized in step a) are all complementary to a same target gene.

13- The method according to claim 12, wherein all the oligonucleotides complementary to a same target gene are complementary to the same strand of said target gene.

14- The method according to claim 1, wherein the oligonucleotide library synthesized in step a) contains oligonucleotides carring mutations allowing introduction of all possible substitutions at each codon of said target gene(s).

15- The method according to claim 1, wherein the oligonucleotide library synthesized in step a) contains oligonucleotides carrying mutations allowing introduction of a same amino acid, preferably an alanine, at each codon of said target gene(s).

16- The method of mutagenesis of a target protein or of several target proteins, characterized in that it comprises preparing a mutant gene expression library from a target gene coding for said protein, or from several target genes coding for said proteins, by the method for producing a mutant gene library according to claim 1, then expressing said mutant genes to produce a library of mutant proteins.
